SEQUENCE LISTING (56 sequences)

SEQ ID NO: 1 - protein, 167 aa, synthetic construct
  REGION 1..167: note = wild type TadA
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 2 - protein, 213 aa, synthetic construct
  REGION 1..213: note = pmCDA1
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAVSRGSG

SEQ ID NO: 3 - protein, 138 aa, synthetic construct
  REGION 1..138: note = DddAtox
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGAIPVKRGATGETKVFTGNSNSPKSPTKGGC

SEQ ID NO: 4 - protein, 7 aa, synthetic construct
  REGION 1..7: note = nuclear localization signal protein
PKKKRKV

SEQ ID NO: 5 - protein, 29 aa, synthetic construct
  REGION 1..29: note = SOD2-MTS
LSRAVCGTSRQLAPVLGYLGSRQKHSLPD

SEQ ID NO: 6 - protein, 28 aa, synthetic construct
  REGION 1..28: note = COX8A-MTS
SVLTPLLLRGLTGSARRLPVPRAKIHSL

SEQ ID NO: 7 - protein, 54 aa, synthetic construct
  REGION 1..54: note = chloroplast transit peptide protein
MDSQLVLSLKLNPSFTPLSPLFPFTPCSSFSPSLRFSSCYSRRLYSPVTVYAAK

SEQ ID NO: 8 - protein, 18 aa, synthetic construct
  REGION 1..18: note = nuclear export signal protein
VDEMTKKFGTLTIHDTEK

SEQ ID NO: 9 - protein, 94 aa, synthetic construct
  REGION 1..94: note = a split of cytosine deaminase (1333C)
PTPYPNYANAGHVEGQSALFMRDNGISEGLVFHNNPEGTCGFCVNMTETLLPENAKMTVVPPEGAIPVKRGATGETKVFTGNSNSPKSPTKGGC

SEQ ID NO: 10 - protein, 44 aa, synthetic construct
  REGION 1..44: note = a split of cytosine deaminase (1333N)
GSYALGPYQISAPQLPAYNGQTVGTFYYVNDAGGLESKVFSSGG

SEQ ID NO: 11 - protein, 30 aa, synthetic construct
  REGION 1..30: note = a split of cytosine deaminase (1397C)
AIPVKRGATGETKVFTGNSNSPKSPTKGGC

SEQ ID NO: 12 - protein, 108 aa, synthetic construct
  REGION 1..108: note = a split of cytosine deaminase (1397N)
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEG

SEQ ID NO: 13 - protein, 564 aa, synthetic construct
  REGION 1..255: note = COX 3.1 Left-mitoTALE
LTPDQVVAIASHDGGKQALETVQRLLPVLCQAHGLTPAQVVAIASHDGGKQALETVQRLLPVLCQDHGLTPAQVVAIASNGGGKQALETVQRLLPVLCQDHGLTPDQVVAIASNIGGKQALETVQRLLPVLCQDHGLTPDQVVAIASHDGGKQALETVQRLLPVLCQDHGLTPAQVVAIASNIGGKQALETVQRLLPVLCQAHGLTPAQVVAIASNIGGKQALETVQRLLPVLCQDHGLTPDQVVAIASNNGGKQALETVQRLLPVLCQDHGLTPEQVVAIASHDGGKQALETVQRLLPVLCQAHGLTPAQVVAIASHDGGKQALETVQRLLPVLCQDHGLTPAQVVAIASNGGGKQALETVQRLLPVLCQDHGLTPAQVVAIASHDGGKQALETVQRLLPVLCQAHGLTPDQVVAIASNIGGKQALETVQRLLPVLCQDHGLTPAQVVAIASNNGGKQALETVQRLLPVLCQDHGLTPDQVVAIASNIGGKQALETVQRLLPVLCQDHGLTPAQVVAIASNNGGKQALETVQRLLPVLCQDHGLTPEQVVAIASNGGGKQALE

SEQ ID NO: 14 - protein, 632 aa, synthetic construct
  REGION 1..255: note = COX 3.1 Right-mitoTALE
LTPDQVVAIASNIGGKQALETVQRLLPVLCQDHGLTPAQVVAIASNNGGKQALETVQRLLPVLCQDHGLTPAQVVAIASNIGGKQALETVQRLLPVLCQAHGLTPAQVVAIASNGGGKQALETVQRLLPVLCQAHGLTPDQVVAIASNNGGKQALETVQRLLPVLCQAHGLTPAQVVAIASHDGGKQALETVQRLLPVLCQAHGLTPDQVVAIASHDGGKQALETVQRLLPVLCQDHGLTPDQVVAIASNNGGKQALETVQRLLPVLCQDHGLTPAQVVAIASNGGGKQALETVQRLLPVLCQAHGLTPEQVVAIASHDGGKQALETVQRLLPVLCQAHGLTPAQVVAIASNNGGKQALETVQRLLPVLCQDHGLTPDQVVAIASNNGGKQALETVQRLLPVLCQDHGLTPDQVVAIASNIGGKQALETVQRLLPVLCQAHGLTPAQVVAIASNIGGKQALETVQRLLPVLCQDHGLTPAQVVAIASNIGGKQALETVQRLLPVLCQAHGLTPEQVVAIASNGGGKQALETVQRLLPVLCQAHGLTPAQVVAIASNNGGKQALETVQRLLPVLCQDHGLTPDQVVAIASNNGGKQALETVQRLLPVLCQDHGLTPEQVVAIASNGGGKQALE

SEQ ID NO: 15 - protein, 496 aa, synthetic construct
  REGION 1..255: note = ND1 Left-mitoTALE
LTPDQVVAIASHDGGKQALETVQRLLPVLCQAHGLTPDQVVAIASNGGGKQALETVQRLLPVLCQDHGLTPAQVVAIASNIGGKQALETVQRLLPVLCQDHGLTPDQVVAIASNNGGKQALETVQRLLPVLCQAHGLTPDQVVAIASHDGGKQALETVQRLLPVLCQDHGLTPDQVVAIASHDGGKQALETVQRLLPVLCQAHGLTPAQVVAIASNGGGKQALETVQRLLPVLCQDHGLTPAQVVAIASNIGGKQALETVQRLLPVLCQAHGLTPDQVVAIASNNGGKQALETVQRLLPVLCQAHGLTPAQVVAIASHDGGKQALETVQRLLPVLCQDHGLTPDQVVAIASHDGGKQALETVQRLLPVLCQAHGLTPEQVVAIASNNGGKQALETVQRLLPVLCQAHGLTPAQVVAIASNGGGKQALETVQRLLPVLCQAHGLTPAQVVAIASNGGGKQALETVQRLLPVLCQAHGLTPEQVVAIASNGGGKQALE

SEQ ID NO: 16 - protein, 598 aa, synthetic construct
  REGION 1..255: note = ND1 Right-mitoTALE
LTPDQVVAIASNNGGKQALETVQRLLPVLCQDHGLTPAQVVAIASNIGGKQALETVQRLLPVLCQAHGLTPEQVVAIASNNGGKQALETVQRLLPVLCQAHGLTPDQVVAIASNGGGKQALETVQRLLPVLCQAHGLTPAQVVAIASNGGGKQALETVQRLLPVLCQAHGLTPEQVVAIASNGGGKQALETVQRLLPVLCQAHGLTPAQVVAIASNNGGKQALETVQRLLPVLCQDHGLTPAQVVAIASNIGGKQALETVQRLLPVLCQAHGLTPEQVVAIASNGGGKQALETVQRLLPVLCQAHGLTPDQVVAIASNNGGKQALETVQRLLPVLCQAHGLTPAQVVAIASHDGGKQALETVQRLLPVLCQDHGLTPDQVVAIASNGGGKQALETVQRLLPVLCQAHGLTPDQVVAIASHDGGKQALETVQRLLPVLCQAHGLTPDQVVAIASNIGGKQALETVQRLLPVLCQAHGLTPAQVVAIASHDGGKQALETVQRLLPVLCQAHGLTPAQVVAIASHDGGKQALETVQRLLPVLCQDHGLTPAQVVAIASHDGGKQALETVQRLLPVLCQAHGLTPEQVVAIASNGGGKQALE

SEQ ID NO: 17 - protein, 564 aa, synthetic construct
  REGION 1..255: note = ND6 Left-mitoTALE
LTPAQVVAIASHDGGKQALETVQRLLPVLCQDHGLTPEQVVAIASNIGGKQALETVQRLLPVLCQAHGLTPAQVVAIASNIGGKQALETVQRLLPVLCQDHGLTPAQVVAIASHDGGKQALETVQRLLPVLCQAHGLTPDQVVAIASHDGGKQALETVQRLLPVLCQDHGLTPDQVVAIASHDGGKQALETVQRLLPVLCQDHGLTPAQVVAIASHDGGKQALETVQRLLPVLCQAHGLTPAQVVAIASNGGGKQALETVQRLLPVLCQDHGLTPDQVVAIASNNGGKQALETVQRLLPVLCQAHGLTPEQVVAIASNIGGKQALETVQRLLPVLCQAHGLTPAQVVAIASHDGGKQALETVQRLLPVLCQAHGLTPAQVVAIASHDGGKQALETVQRLLPVLCQDHGLTPAQVVAIASHDGGKQALETVQRLLPVLCQDHGLTPAQVVAIASHDGGKQALETVQRLLPVLCQAHGLTPAQVVAIASHDGGKQALETVQRLLPVLCQDHGLTPDQVVAIASNIGGKQALETVQRLLPVLCQAHGLTPEQVVAIASNGGGKQALE

SEQ ID NO: 18 - protein, 598 aa, synthetic construct
  REGION 1..255: note = ND6 Right-mitoTALE
LTPEQVVAIASNIGGKQALETVQRLLPVLCQAHGLTPDQVVAIASHDGGKQALETVQRLLPVLCQAHGLTPDQVVAIASNGGGKQALETVQRLLPVLCQDHGLTPDQVVAIASNIGGKQALETVQRLLPVLCQDHGLTPDQVVAIASHDGGKQALETVQRLLPVLCQDHGLTPAQVVAIASNIGGKQALETVQRLLPVLCQAHGLTPAQVVAIASNNGGKQALETVQRLLPVLCQAHGLTPDQVVAIASHDGGKQALETVQRLLPVLCQDHGLTPAQVVAIASNNGGKQALETVQRLLPVLCQAHGLTPEQVVAIASNIGGKQALETVQRLLPVLCQAHGLTPAQVVAIASNGGGKQALETVQRLLPVLCQAHGLTPAQVVAIASNNGGKQALETVQRLLPVLCQDHGLTPDQVVAIASNNGGKQALETVQRLLPVLCQAHGLTPAQVVAIASHDGGKQALETVQRLLPVLCQDHGLTPDQVVAIASNGGGKQALETVQRLLPVLCQAHGLTPEQVVAIASNIGGKQALETVQRLLPVLCQAHGLTPDQVVAIASNGGGKQALETVQRLLPVLCQDHGLTPEQVVAIASNGGGKQALE

SEQ ID NO: 19 - DNA, 19 bp, synthetic construct
  misc_feature 1..19: note = human mtWGS 1 1st PCR forward primer
  source 1..19: mol_type = other DNA
gcaaatcttaccccgcctg

SEQ ID NO: 20 - DNA, 20 bp, synthetic construct
  misc_feature 1..20: note = human mtWGS 1 1st PCR reverse primer
  source 1..20: mol_type = other DNA
aattaggctgtgggtggttg

SEQ ID NO: 21 - DNA, 20 bp, synthetic construct
  misc_feature 1..20: note = human mtWGS 2 1st PCR forward primer
  source 1..20: mol_type = other DNA
gccatactagtctttgccgc

SEQ ID NO: 22 - DNA, 19 bp, synthetic construct
  misc_feature 1..19: note = human mtWGS 2 1st PCR reverse primer
  source 1..19: mol_type = other DNA
ggcaggtcaatttcactgg

SEQ ID NO: 23 - DNA, 20 bp, synthetic construct
  misc_feature 1..20: note = human mtWGS 1 2nd PCR forward primer (COX 3.1)
  source 1..20: mol_type = other DNA
tcaatcacctgagctcacca

SEQ ID NO: 24 - DNA, 20 bp, synthetic construct
  misc_feature 1..20: note = human mtWGS 1 2nd PCR reverse primer (COX 3.1)
  source 1..20: mol_type = other DNA
attagttggcggatgaagca

SEQ ID NO: 25 - DNA, 22 bp, synthetic construct
  misc_feature 1..22: note = human mtWGS 1 2nd PCR forward primer (ND1)
  source 1..22: mol_type = other DNA
aagggtggagaggttaaaggag

SEQ ID NO: 26 - DNA, 20 bp, synthetic construct
  misc_feature 1..20: note = human mtWGS 1 2nd PCR reverse primer (ND1)
  source 1..20: mol_type = other DNA
ccctggtcaacctcaaccta

SEQ ID NO: 27 - DNA, 20 bp, synthetic construct
  misc_feature 1..20: note = human mtWGS 2 2nd PCR forward primer (ND6)
  source 1..20: mol_type = other DNA
ccacagcaccaatcctacct

SEQ ID NO: 28 - DNA, 20 bp, synthetic construct
  misc_feature 1..20: note = human mtWGS 2 2nd PCR reverse primer (ND6)
  source 1..20: mol_type = other DNA
tgattgttagcggtgtggtc

SEQ ID NO: 29 - DNA, 20 bp, synthetic construct
  misc_feature 1..20: note = 1st PCR forward primer (TYRO3)
  source 1..20: mol_type = other DNA
ctgtcaacaaagtgctggcc

SEQ ID NO: 30 - DNA, 20 bp, synthetic construct
  misc_feature 1..20: note = 1st PCR reverse primer (TYRO3)
  source 1..20: mol_type = other DNA
cttgactcccatgcctcctg

SEQ ID NO: 31 - DNA, 18 bp, synthetic construct
  misc_feature 1..18: note = 2nd PCR forward primer (TYRO3)
  source 1..18: mol_type = other DNA
gtcaacaaagtgctggcc

SEQ ID NO: 32 - DNA, 19 bp, synthetic construct
  misc_feature 1..19: note = 2nd PCR reverse primer (TYRO3)
  source 1..19: mol_type = other DNA
ttgactcccatgcctcctg

SEQ ID NO: 33 - DNA, 20 bp, synthetic construct
  misc_feature 1..20: note = 1st PCR forward primer (OT1)
  source 1..20: mol_type = other DNA
cctgaccgtgctaactgtgt

SEQ ID NO: 34 - DNA, 20 bp, synthetic construct
  misc_feature 1..20: note = 1st PCR reverse primer (OT1)
  source 1..20: mol_type = other DNA
ttcatgctcattccccccac

SEQ ID NO: 35 - DNA, 20 bp, synthetic construct
  misc_feature 1..20: note = 1st PCR forward primer (OT2)
  source 1..20: mol_type = other DNA
ggcggtacaagaacaacgag

SEQ ID NO: 36 - DNA, 20 bp, synthetic construct
  misc_feature 1..20: note = 1st PCR reverse primer (OT2)
  source 1..20: mol_type = other DNA
ggggacacacacagagaacc

SEQ ID NO: 37 - DNA, 22 bp, synthetic construct
  misc_feature 1..22: note = 1st PCR forward primer (OT3)
  source 1..22: mol_type = other DNA
aagtccctggatcatattcaca

SEQ ID NO: 38 - DNA, 19 bp, synthetic construct
  misc_feature 1..19: note = 1st PCR reverse primer (OT3)
  source 1..19: mol_type = other DNA
ggatcttctgagcgggtgt

SEQ ID NO: 39 - DNA, 20 bp, synthetic construct
  misc_feature 1..20: note = 1st PCR forward primer (OT4)
  source 1..20: mol_type = other DNA
ggtgacaacagagcaaagca

SEQ ID NO: 40 - DNA, 21 bp, synthetic construct
  misc_feature 1..21: note = 1st PCR reverse primer (OT4)
  source 1..21: mol_type = other DNA
agtacatttccccgctgataa

SEQ ID NO: 41 - DNA, 20 bp, synthetic construct
  misc_feature 1..20: note = 1st PCR forward primer (OT5)
  source 1..20: mol_type = other DNA
ggctttgaccagcaaccaag

SEQ ID NO: 42 - DNA, 20 bp, synthetic construct
  misc_feature 1..20: note = 1st PCR reverse primer (OT5)
  source 1..20: mol_type = other DNA
tggaccacactgacaggaga

SEQ ID NO: 43 - DNA, 20 bp, synthetic construct
  misc_feature 1..20: note = 1st PCR forward primer (OT6)
  source 1..20: mol_type = other DNA
agccaagacctcacacacac

SEQ ID NO: 44 - DNA, 20 bp, synthetic construct
  misc_feature 1..20: note = 1st PCR reverse primer (OT6)
  source 1..20: mol_type = other DNA
aaaacacagccccagagagg

SEQ ID NO: 45 - DNA, 22 bp, synthetic construct
  misc_feature 1..22: note = 2nd PCR forward primer (OT1)
  source 1..22: mol_type = other DNA
ggagcttttaggatgtgggagt

SEQ ID NO: 46 - DNA, 20 bp, synthetic construct
  misc_feature 1..20: note = 2nd PCR reverse primer (OT1)
  source 1..20: mol_type = other DNA
agggaaagggagacttggga

SEQ ID NO: 47 - DNA, 20 bp, synthetic construct
  misc_feature 1..20: note = 2nd PCR forward primer (OT2)
  source 1..20: mol_type = other DNA
cggctcaaggagaaccagat

SEQ ID NO: 48 - DNA, 20 bp, synthetic construct
  misc_feature 1..20: note = 2nd PCR reverse primer (OT2)
  source 1..20: mol_type = other DNA
agggaacagggcgtaagtct

SEQ ID NO: 49 - DNA, 21 bp, synthetic construct
  misc_feature 1..21: note = 2nd PCR forward primer (OT3)
  source 1..21: mol_type = other DNA
caggatgtcctgaaattaggg

SEQ ID NO: 50 - DNA, 20 bp, synthetic construct
  misc_feature 1..20: note = 2nd PCR reverse primer (OT3)
  source 1..20: mol_type = other DNA
tggcgatctcgtagaacaca

SEQ ID NO: 51 - DNA, 20 bp, synthetic construct
  misc_feature 1..20: note = 2nd PCR forward primer (OT4)
  source 1..20: mol_type = other DNA
atcacactaccccaccctca

SEQ ID NO: 52 - DNA, 20 bp, synthetic construct
  misc_feature 1..20: note = 2nd PCR reverse primer (OT4)
  source 1..20: mol_type = other DNA
atcttccttcgacccccaga

SEQ ID NO: 53 - DNA, 20 bp, synthetic construct
  misc_feature 1..20: note = 2nd PCR forward primer (OT5)
  source 1..20: mol_type = other DNA
tttacagtggcttccgtggg

SEQ ID NO: 54 - DNA, 20 bp, synthetic construct
  misc_feature 1..20: note = 2nd PCR reverse primer (OT5)
  source 1..20: mol_type = other DNA
tggaccacactgacaggaga

SEQ ID NO: 55 - DNA, 20 bp, synthetic construct
  misc_feature 1..20: note = 2nd PCR forward primer (OT6)
  source 1..20: mol_type = other DNA
gacgcttgctctgtttgtgg

SEQ ID NO: 56 - DNA, 20 bp, synthetic construct
  misc_feature 1..20: note = 2nd PCR reverse primer (OT6)
  source 1..20: mol_type = other DNA
aaaacacagccccagagagg

(19) United States
(12) Patent Application Publication
(10) Pub. No.: US 2026/0337590 A1
(43) Pub. Date: Oct. 29, 2026

(54) NOVEL ADENINE DEAMINASE VARIANTS AND A METHOD FOR BASE EDITING USING THE SAME

(71) Applicant: INSTITUTE FOR BASIC SCIENCE, Daejeon (KR)

(72) Inventors: Jin-Soo KIM, Seoul (KR); Sung Ik CHO, Seoul (KR); Kayeong LIM, Seoul (KR); Jaesuk LEE, Seoul (KR)

(21) Appl. No.: 19/114,082

(22) PCT Filed: Jul. 4, 2023
(86) PCT No.: PCT/KR2023/009361

§ 371 Date: Mar. 21, 2025

(30) Foreign Application Priority Data
Sep. 23, 2022 (KR) 10-2022-0120697

(51) Int. Cl. C12N 9/78; A61K 48/00; C12N 9/22; C12N 15/10; C12N 15/63

(52) U.S. Cl. CPC C12N 9/78; A61K 48/005; C12N 9/222; C12N 15/102; C12N 15/63; C12Y 305/04001; C12Y 305/04002

(57) ABSTRACT

The present invention relates a novel adenine deaminase variant, a fusion protein comprising the adenine deaminase variant, a base editing composition for A-to-G base editing in DNA comprising the fusion protein, and a method for A-to-G base editing in DNA comprising delivering the base editing composition to a cell containing a target DNA. The novel adenine deaminase variant can result in remarkably reduced off-target effects involving an unintended base alteration in DNA and/or RNA, and induce base editing only at a single nucleotide residue without any unintended off-target editing in the target DNA.

### REFERENCE TO AN ELECTRONIC SEQUENCE LISTING

The contents of the electronic sequence listing (FPT20253129US_SEQ.xml; Size: 74,351 bytes; and Date of Creation: Mar. 6, 2025) is herein incorporated by reference in its entirety. The contents of the electronic sequence listing in no way introduces new matter into the specification.

### TECHNICAL FIELD

Provided are: an adenine deaminase variant capable of reducing off-target editing; a fusion protein comprising a DNA-binding protein, and the adenine deaminase variant; a base editing composition for A-to-G base editing in DNA comprising the fusion protein; and a method for A-to-G base editing in DNA comprising delivering the base editing composition to a cell containing a target DNA.

### BACKGROUND ART

Targeted base editing of mammalian mitochondrial DNA (mtDNA) is a powerful and versatile technology, which can be used to model mitochondrial genetic diseases in cell lines and animals and to develop novel therapeutic modalities that correct pathogenic mutations in patients. Programmable deaminases, which are composed of a custom DNA-binding protein and a nucleobase deaminase, enable mitochondrial DNA editing in a targeted manner.

In contrast to cytosine base editors, adenine base editors, known as TALEDs, incorporate TadA8e, a deoxy-adenine deaminase engineered from the tRNA-specific TadA protein derived from E. coli. TadA8e and related variants of TadA are crucial components in CRISPR RNA-guided adenine base editors, extensively employed for A-to-G base editing of nuclear DNA. Nevertheless, the use of RNA-guided ABEs for editing organellar DNA poses a challenge due to the difficulty of delivering guide RNA into organelles. Furthermore, TadASe present in ABEs retains residual deaminase activity for RNA substrates, resulting in unintended, transcriptome-wide off-target base editing. Off-target effects arise when the editing machinery mistakenly acts on unintended genomic regions, leading to undesired modifications.

Despite the widespread interest in programmable deaminase-mediated base editing, there currently lack developed strategies for adenine base editors with enhanced specificity, thereby reducing the occurrence of off-target editing effects. The development of such base editors that minimize these side effects and the implementation of methods to reduce them hold substantial importance, rendering them more reliable and applicable to a broader range of scientific and therapeutic applications.

### SUMMARY

The present invention relates to a base editing composition and its method of application in gene therapy and genome engineering. More specifically, the composition and method involve base editor variants that exhibit a significant reduction in off-target genome cleavage and side effects, such as RNA off-target toxicity, when employed in clinical settings. By introducing innovative adenine base editors and an accompanying novel utilization approach, the present invention offers a fresh avenue for targeted base editing with wide-ranging applications in the fields of medicine and biotechnology.

In one embodiment, provided herein is an adenine deaminase variant.

In another embodiment, provided herein is a fusion protein comprising a DNA-binding protein, and the adenine deaminase variant.

In yet another embodiment, provided herein is a polynucleotide encoding the adenine deaminase variant or the fusion protein, and an expression vector comprising the polynucleotide.

In one embodiment, described herein is a base editing composition for A-to-G base editing in DNA comprising the fusion protein, a polynucleotide encoding the fusion protein or an expression vector comprising the polynucleotide.

In yet another embodiment, provided herein is a method for A-to-G base editing in DNA comprising delivering the base editing composition to a cell containing a target DNA.

In another embodiment, described herein is a method for reducing off-target editing effects, the method comprising delivering the base editing composition to a cell containing a target DNA.

In yet another embodiment, provided is a use of the adenine deaminase variant or the fusion protein in A-to-G base editing in DNA and/or reducing off-target effect in A-to-G base editing in DNA, or in preparing a composition for A-to-G base editing in DNA and/or reducing off-target effect in A-to-G base editing in DNA.

### BRIEF DESCRIPTION OF DRAWINGS

FIG. 1(a) exemplifies the structures of base editors used in the present invention. AD (TadA8e adenine deaminase); AD* (TadA8e adenine deaminase variant); MTS (mitochondrial targeting sequence); UGI (uracil glycosylase inhibitor).

FIG. 1(b) is a graph showing the number of RNA edits and editing frequencies for the Cox3.1-specific sTALEDs.

FIG. 1(c) is graph showing A-to-G edits and C-to-T edits for the Cox3.1-specific STALEDs (left), as well as on-target activity of Cox3.1-specific sTALED variants relative to that of the wild-type Cox3.1-specific sTALED (right).

FIG. 1(d) is a graph showing the number of RNA edits and editing frequencies for the ND1-specific sTALEDs.

FIG. 1(e) is graphs showing A-to-G edits and C-to-T edits for the ND1-specific sTALEDs (left), as well as on-target activity of the ND1-specific sTALED variants relative to that of the wild-type ND1-specific sTALED (right).

FIG. 2(a) Structural representations of the TadA portion of ABE8e (Protein Data Bank (PDB) accession number 6VPC).

FIG. 2(b) is a heat map showing DNA on-target activity (left), RNA off-target activity (middle), and the relative ratio of DNA on-target editing frequencies to RNA off-target editing frequencies (right) of the 101 sTALED variants among the total of 209 sTALED variants that retain mitochondrial DNA on-target activity. The relative ratio is normalized to that for the original STALED, which has a value of 1.

FIG. 3 is graphs showing DNA on-target base editing frequencies induced by 209 STALED variants.

FIG. 4(a) is a graph showing the editing frequencies at six RNA off-target sites measured by targeted RNA sequencing.

FIG. 4(b) is a graph showing RNA off-target editing frequencies induced by sTALED and STALED variants extracted from transcriptome-wide sequencing at six selected RNA off-target sites.

FIG. 4(c) is a graph showing RNA off-target editing frequencies induced by sTALED and STALED variants analyzed by targeted RNA amplicon sequencing.

FIG. 5(a) is a graph showing the number of RNA edits and editing frequencies for the Cox3.1-specific sTALEDs.

FIG. 5(b) is graphs showing A-to-G edits and C-to-T edits for the Cox3.1-specific STALEDs.

FIG. 5(c) is a graph showing the number of RNA edits and editing frequencies for the ND1-specific sTALEDs.

FIG. 5(d) is graphs showing A-to-G edits and C-to-T edits for the ND1-specific sTALEDs.

FIG. 5(e) is a graph showing the number of RNA edits and editing frequencies for the ND6-specific sTALEDs.

FIG. 5(f) is a graph showing A-to-G edits and C-to-T edits for the ND6-specific sTALEDs.

FIG. 6(a) is a graph showing on-target activities of ND1-specific sTALED variants relative to that of wild-type ND1-specific sTALED.

FIG. 6(b) is a graph showing on-target activities of ND6-specific sTALED variants relative to that of wild-type ND6-specific sTALED.

FIG. 6(c) is a heat map showing RNA off-target activities of ND1-specific sTALED and STALED variants at six representative sites.

FIG. 6(d) is a heat map showing RNA off-target activities of ND6-specific sTALED and STALED variants at six representative sites.

FIG. 6(e) is a bar graph showing the frequencies of RNA off-target edits induced by ND1-specific sTALED and sTALED variants at six representative sites. The editing efficiency of each replicate is displayed on the graph as a dot. Error bars are s.e.m. for n=2 (replicates)×6 (RNA off-target sites) biologically independent samples.

FIG. 6(f) is a bar graph showing the frequencies of RNA off-target edits induced by ND6-specific sTALED and sTALED variants at six sites. The efficiency of each replicate is displayed on the graph as a dot. Error bars are s.e.m. for n=2 (replicates)×6 (RNA off-target sites) biologically independent samples.

FIG. 6(g) is a bar graph showing the ratio of DNA on-target editing frequencies relative to RNA off-target editing frequencies induced by ND1-specific sTALED and sTALED variants.

FIG. 6(h) is a bar graph showing the ratio of DNA on-target editing frequencies relative to RNA off-target editing frequencies induced by ND6-specific sTALED and sTALED variants. This ratio is normalized to that for sTALED, which has a value of 1.

FIG. 6(i) is a graph showing average relative ratio values for the sTALEDs and sTALED variants targeted to Cox3.1, ND1 (FIG. 6(g)), and ND6 (FIG. 6(h)).

FIG. 7(a) is a heat maps depicting A-to-G conversions caused by sTALED or sTALED variants targeted to the Cox3.1 site.

FIG. 7(b) is a heat maps depicting A-to-G conversions caused by sTALED or sTALED variants targeted to the ND1 site.

FIG. 7(c) is a heat maps depicting A-to-G conversions caused by sTALED or sTALED variants targeted to the ND6 site.

FIGS. 7(d)-(f) show the analysis of Cox3.1, ND1, ND6 alleles summarized in FIGS. 7(a)-(c), respectively. The spacer sequence is shown on the left, and bar graphs displaying the frequency of each allele are shown on the right. The reference sequence is written all in capital letters, whereas the lowercase letters indicate the positions at which base editing has taken place.

FIG. 8(a) shows the plots indicating the positions of on-target and off-target edits across the mitochondrial genome at day 4 post-transfection. Black, and gray dots indicate off-target edits, and naturally-occurring single-nucleotide variations (SNVs), respectively, and the arrows indicate on-target (and bystander) edits. Nucleotide positions in the human mitochondrial genome are represented on the X axis.

FIG. 8(b) shows the average frequencies of genome-wide off-target edits induced by wild-type TALED and TALED variants.

FIG. 9(a) shows the plots indicating the positions of on-target and off-target edits across the mitochondrial genome at day 2 post-transfection. Black, and gray dots indicate off-target edits, and naturally-occurring single-nucleotide variations (SNVs), respectively, and the arrows indicate on-target (and bystander) edits. Nucleotide positions in the human mitochondrial genome are represented on the X axis.

FIG. 9(b) is a graph showing the average frequencies of genome-wide off-target edits induced by wild-type TALED and TALED variants. Error bars are s.e.m. for n=2 biologically independent samples.

FIGS. 10(a)-(c) are line graphs showing frequencies of on-target base edits induced by Cox3.1-(a), ND1-(b), and ND6-specific (c) sTALEDs and sTALED variants (V106W, V28R, R111S) over time. Error bars are s.e.m. for n=2 biologically independent samples.

FIGS. 10(d)-(f) are line graphs showing frequencies of RNA off-target base edits induced by Cox3.1-(d), ND1-(e), and ND6-specific (f) sTALEDs and sTALED variants (V106W, V28R, R111S) at six representative sites over time. Error bars are s.e.m. for n=2 biologically independent samples.

FIG. 10(g) is a line graph showing average RNA off-target editing frequencies for Cox3.1-, ND1-, and ND6-specific sTALEDs and sTALED variants over time. Error bars are s.e.m. for n=2 biologically independent samples.

FIG. 11(a) illustrated an experimental scheme of one embodiment of the present invention.

FIGS. 11(b) and(c) are bar graphs showing viability of cells transfected with plasmids expressing sTALED, STALED-V106W, STALED-V28R and sTALED-R111S targeted to the indicated sites which was determined by observing the color change caused by formazan formation in an MTS assay at day 2 (B) and day 4 (C) post-transfection. The absorbance values were normalized to those of cells transfected with pEGFP as a control. Error bars are s.e.m. for n=2 biologically independent samples.

FIG. 12(a) exemplifies the architecture of ABE8e and ABE8e variant constructs. AD (TadA8e adenine deaminase); AD* (TadA8e adenine deaminase variant); NLS (nuclear localization sequence).

FIG. 12(b) is a graph showing on-target activity of ABE8e and the ABE8e variants (ABE8e-V106W, ABE8e-V28R, ABE8e-R111S) at the nuclear TYRO3 site.

FIG. 12(c) depicts a heat map showing the frequencies of A-to-G conversions caused by ABE8e and the ABE8e variants (ABE8e-V106W, ABE8e-V28R, ABE8e-R111S) at the nuclear TYRO3 site.

FIG. 12(d) is a graph showing RNA off-target activity of TYRO3-targeted ABE8e and ABE8e variants (ABE8e-V106W, ABE8e-V28R, ABE8e-R111S) at six representative sites.

FIGS. 12(e) and 12(f) are graphs illustrating the total number of RNA edits found in HEK 293T cells that expressed ABE8e or the ABE8e variants targeted to the nuclear TYRO3 site as assessed by whole transcriptome sequencing.

FIG. 13(a) is a graph showing DNA on-target activity of Cox3-specific TALEDs including dimeric TALEDs (dTALEDs), half monomer (dTALED-ADs), monomeric TALEDS (mTALEDs), and untreated samples.

FIG. 13(b) is a graph showing RNA off-target activity of Cox3-specific TALEDs including dimeric TALEDs (dTALEDs), half monomer (dTALED-ADs), monomeric TALEDS (mTALEDs), and untreated samples at six representative sites.

FIG. 13(c) is a graph showing specificity ratios of RNA off-target editing relative to on-target editing induced by Cox3-specific TALEDs.

### DETAILED DESCRIPTION OF THE INVENTION

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting. Unless defined otherwise, all technical and scientific terms used herein have the meaning commonly understood by a person skilled in the art to which this invention belongs.

In this application, the use of the singular includes the plural unless specifically stated otherwise. It must be noted that, as used in the specification, the singular forms “a,” “an,” and “the” include plural references unless the context clearly dictates otherwise. In this application, the use of “or” means “and/or,” unless stated otherwise, and is understood to be inclusive. Furthermore, use of the term “including” as well as other forms, such as “include,” “includes,” and “included,” is not limiting.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, such as within 5-fold or within 2-fold, of a value. Where particular values are described in the application and claims, unless otherwise stated the term “about” meaning within an acceptable error range for the particular value should be assumed.

As used herein, the term “corresponding” or “corresponds” refers to amino acid residues at positions listed in the polypeptide or amino acid residues that are similar, identical, or homologous to those listed in the polypeptide. Identifying the amino acid at the corresponding position may be determining a specific amino acid in a sequence that refers to a specific sequence. As used herein, “corresponding region” generally refers to a similar or corresponding position in a related protein or a reference protein. For example, an arbitrary amino acid sequence is aligned with SEQ ID NO: 3, and based on this, each amino acid residue of the amino acid sequence may be numbered with reference to the amino acid residue of SEQ ID NO: 3 and the numerical position of the corresponding amino acid residue. For example, a sequence alignment algorithm as described in the present disclosure may determine the position of an amino acid or a position at which modification such as substitution, insertion, or deletion occurs through comparison with that in a query sequence (also referred to as a “reference sequence”)

As used herein, the term “alignment” means mapping sequence reads to a reference genome and then aligning the bases having identical sites in genomes to fit for each site. Accordingly, so long as it can align sequence reads in the same manner as above, any computer program may be employed. The program may be one already known in the pertinent art or may be selected from among programs tailored to the purpose. In one embodiment, alignment is performed using ISAAC, but is not limited thereto.

Reference in the specification to “various embodiments,” “some embodiments,” “an embodiment,” “one embodiment” or “other embodiments” means that a particular feature, structure, or characteristic described in connection with the embodiments is included in at least some embodiments, but not necessarily all embodiments, of the present disclosures.

As used herein, the term “host cell” (or “recombinant host cell”), as used herein, is intended to refer to a cell that has been genetically altered, or is capable of being genetically altered by introduction of an exogenous polynucleotide molecule, such as a recombinant plasmid or vector. It should be understood that such terms are intended to refer not only to the particular subject cell but to the progeny of such a cell. Because certain modifications may occur in succeeding generations due to either mutation or environmental influences, such progency may not, in fact, be identical to the parent cell, but are still included within the scope of the term “host cell” as used herein.

As used herein, the expression “base editor (BE)” is meant an agent that binds a polynucleotide and has nucleobase modifying activity. In one embodiment, the base editor comprises a nucleobase modifying polypeptide (e.g., a deaminase) and a nucleic acid programmable nucleotide binding domain. In another embodiment, the base editor comprises a nucleobase modifying polypeptide (e.g., a deaminase) and a nucleic acid programmable nucleotide binding domain in conjunction with a guide polynucleotide (e.g., guide RNA). Yet, in another embodiment, the agent is a biomolecular complex comprising a protein domain having base editing activity, i.e., a domain capable of modifying a base (e.g., A, T, C, G, I, or U) within a nucleic acid molecule (e.g., DNA). In some embodiments, the polynucleotide programmable DNA binding domain is fused or linked to a deaminase domain. In one embodiment, the agent is a fusion protein comprising a domain having base editing activity. In another embodiment, the protein domain having base editing activity is linked to the guide RNA (e.g., via an RNA binding motif on the guide RNA and an RNA binding domain fused to the deaminase). In some embodiments, the domain having base editing activity is capable of deaminating a base within a nucleic acid molecule. In some embodiments, the base editor is capable of deaminating one or more bases within a DNA molecule. In some embodiments, the base editor is capable of deaminating an adenine (A) within DNA. In some embodiments, the base editor is an adenine base editor (ABE).

As used herein, “administering” is referred to herein as providing one or more compositions described herein to a patient or a subject. By way of example and without limitation, composition administration, e.g., injection, can be performed by intravenous (i.v.) injection, sub-cutaneous (s.c.) injection, intradermal (i.d.) injection, intraperitoneal (i.p.) injection, or intramuscular (i.m.) injection. One or more such routes can be employed. Parenteral administration can be, for example, by bolus injection or by gradual perfusion over time. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrastemally. Alternatively, or concurrently, administration can be by the oral route.

As used herein, the expression “another amino acid” may be intended to refer to an amino acid selected from among alanine, isoleucine, leucine, methionine, phenylalanine, proline, tryptophan, valine, asparagine, cysteine, glutamine, glycine, serine, threonine, tyrosine, aspartic acid, glutamic acid, arginine, histidine, lysine, and all known variants thereof, exclusive of the amino acid having a wild-type protein retained at the original substitution position.

As used herein, the term “off-target site” may refer to a site that is not an on-target site, but to which the adenine base editors show activity. That is, the off-target site may refer to a site where base editing occurs, besides an on-target site. In an embodiment, the term “off-target site” may be used to cover not only sites that are not on-target sites of the adenine base editors, but also sites having possibility to be off-target sites thereof.

As used herein, the term “whole genome sequencing” (WGS) refers to a method of reading the genome by many multiples such as in 10×, 20×, and 40× formats for whole genome sequencing by next generation sequencing. The term “Next generation sequencing” means a technology that fragments the whole genome or targeted regions of genome in a chip-based and PCR-based paired end format and performs sequencing of the fragments by high throughput on the basis of chemical reaction (hybridization).

The term “nucleic acid”, as used herein, refers to either DNA or RNA. “Nucleic acid sequence” or “polynucleotide sequence” refers to a single- or double-stranded polymer of deoxyribonucleotide or ribonucleotide bases read from the 5′ to the 3′ end. It includes both self-replicating plasmids, infectious polymers of DNA or RNA, and nonfunctional DNA or RNA.

The phrase “nucleic acid molecule encoding” refers to a nucleic acid molecule which directs the expression of a specific protein or peptide. The nucleic acid sequences include both the DNA strand sequence that is transcribed into RNA and the RNA sequence that is translated into protein or peptide. The nucleic acid molecule includes both the full-length nucleic acid sequences as well as non-full length sequences derived from the full length protein. It being further understood that the sequence includes the degenerate codons of the native sequence or sequences which may be introduced to provide codon preference in a specific host cell.

The term “vector”, refers to viral expression systems, autonomous self-replicating circular DNA (plasmids), and includes both expression and nonexpression plasmids. Where a recombinant microorganism or cell is described as hosting an “expression vector,” this includes both extrachromosomal circular DNA and DNA that has been incorporated into the host chromosome(s). Where a vector is being maintained by a host cell, the vector may either be stably replicated by the cells during mitosis as an autonomous structure, or the vector may be incorporated within the host's genome.

The term “plasmid” refers to an autonomous circular DNA molecule capable of replication in a cell, and includes both the expression and nonexpression types. Where a recombinant microorganism or cell is described as hosting an “expression plasmid”, this includes latent viral DNA integrated into the host chromosome(s). Where a plasmid is being maintained by a host cell, the plasmid is either being stably replicated by the cell during mitosis as an autonomous structure, or the plasmid is incorporated within the host's genome.

As used herein, the “percentage amino acid sequence homology” or percent amino acid sequence identity” refers to between a first amino acid sequence and a second amino acid sequence. may be calculated by dividing [the number of amino acid residues in the first amino acid sequence that are identical to the amino acid residues at the corresponding positions in the second amino acid sequence] by [the total number of amino acid residues in the first amino acid sequence] and multiplying by [100%], in which each deletion, insertion, substitution or addition of an amino acid residue in the second amino acid sequence-compared to the first amino acid sequence-is considered as a difference at a single amino acid residue (position), i.e. as an “amino acid difference” as defined herein. Alternatively, the degree of sequence identity between two amino acid sequences may be calculated using a known computer algorithm, such as those mentioned above for determining the degree of sequence identity for nucleotide sequences, again using standard settings.

In various embodiments of the present invention, to address the issue of unwanted off-target effects associated with TadA8e, a process of selecting TadA8e variants with reduced RNA off-target effects or DNA off-target effects (including, for example, bystander off-target effects) were delineated. In one embodiment, such objective was accomplished by substituting different amino acid residues at specific locations within TadA8e that interact with nucleotides. Further, to evaluate the impact of these variants on RNA or DNA off-target effects, RNA sequencing as well as whole mitochondrial genome sequencing were performed. This comprehensive analysis allowed for the identification and confirmation of either the entire spectrum of RNA off-target sites or a representative selection of six prominent RNA off-target sites. In addition, recognizing that RNA has transient characteristic, the dynamics of RNA off-target effects over time were also measured. Such measurements were taken at various time points to assess how the RNA off-target landscape changed over the course of expression.

In one embodiment, provided is an adenine deaminase comprising the amino acid sequence of SEQ ID NO: 1 or an amino acid sequence having at least 80% sequence homology to the amino acid sequence of SEQ ID NO:1, wherein at least one amino acid residue selected from residues 28, 30, 46, 48, 49, 82, 84, 106, 108, 110, and 111 of SEQ ID NO:1 or the corresponding amino acid residue of the amino acid sequence having at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence homology (or sequence identity) to the amino acid sequence of SEQ ID NO: 1 is substituted with another amino acid.

The term “adenine deaminase” refers to a polypeptide or a fragment capable of catalyzing the hydrolytic deamination of adenine or adenosine. In one embodiment, the deaminase or deaminase domain represents an adenine deaminase that facilitates the hydrolytic deamination of adenosine to inosine or deoxyadenosine to deoxyinosine. Additionally, in another embodiment, the adenine deaminase performs the hydrolytic deamination of adenine or adenosine in DNA (deoxyribonucleic acid). The adenosine deaminases, such as engineered adenosine deaminases or evolved adenosine deaminases, described herein can be derived from any organism, including bacteria.

In some embodiments, the adenosine deaminase is a TadA deaminase. In some embodiments, the TadA deaminase is TadA variant. In some embodiments, the TadA variant is a TadASe. In some embodiments, the deaminase or deaminase domain is a variant of a naturally occurring deaminase from an organism, such as bacteria, archaea, human, chimpanzee, gorilla, monkey, cow, dog, rat, or mouse. In some embodiments, the deaminase or deaminase domain does not occur in nature. For example, in some embodiments, the deaminase or deaminase domain is at least 50%, at least 55%, at least 60%, at least 65%, at least 70%, at least 75% at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to a naturally occurring deaminase

TadASe adenosine deaminase has the following sequence:

(SEQ ID NO: 1)
MSEVEFSHEYWMRHALTLAKRARDEREVPVGAVLVLNNRVIGEGW
NRAIGLHDPTAHAEIMALRQGGLVMQNYRLIDATLYVTFEPCVMC
AGAMIHSRIGRVVFGVRNSKRGAAGSLMNVLNYPGMNHRVEITEG
ILADECAALLCDFYRMPRQVFNAQKKAQSSIN

In some embodiments, the amino acid substitution may be at least one selected from the group consisting of V28Q, or V28R, A48W, F84M, V106A, K110S, K110T, or K110V, and R111F, R111Q, R111S, R11IT, or R111Y of the amino acid sequence of SEQ ID NO:1.

In one embodiment, the amino acid substitution having the lowest RNA or DNA off-target editing efficiency (including, for example, bystander off-target effects) may be at least one selected from the group consisting of V28Q, or V28R, A48W, and R111S, of the amino acid sequence of SEQ ID NO:1.

In various embodiments, the adenine deaminase variant may exhibit remarkably reduced off-target effects involving an unintended base alteration in DNA and/or RNA. In another embodiment, the adenine deaminase variant may reduce unwanted bystander effects while narrowing activity windows. Alternatively, the adenine deaminase variant may induce base editing only at a single nucleotide residue without any intended off-target editing in the target DNA with a frequency of at least 10%, at least 11%, at least 12%, at least 13%, at least 14%, or at least 15%.

In one embodiment, provided is a fusion protein comprising a DNA-binding protein; and the adenine deaminase variant. The amino acid substitution of the adenine deaminase variant may be at least one selected from the group consisting of V28Q, or V28R, A48W, F84M, V106A, K110S, K110T, or K110V, and R111F, R111Q, R111S, R11IT, or R111Y of the amino acid sequence of SEQ ID NO:1. In one embodiment, the amino acid substitution of the adenine deaminase variant having the lowest RNA or DNA off-target editing effects (including, for example, bystander off-target effects) may be at least one selected from the group consisting of V28Q, or V28R, A48W, and R111S, of the amino acid sequence of SEQ ID NO:1.

In various embodiments, the fusion protein may exhibits remarkably reduced off-target effects involving an unintended base alteration in DNA and/or RNA. In another embodiment, the fusion protein may reduce unwanted bystander effects while narrowing activity windows. Alternatively, the fusion protein may induce base editing only at a single nucleotide residue without any intended off-target editing in the target DNA with a frequency of at least 10%, at least 11%, at least 12%, at least 13%, at least 14%, or at least 15%.

The DNA binding protein may be, for example, but is not limited to, 1) zinc finger protein, 2) transcriptional activator-like effector (TALE) protein, 3) CRISPR-associated nuclease. The nuclease is a type II and/or type V such as Cas protein (e.g., Cas9 protein (CRISPR (Clustered regularly interspaced short palindromic repeats) associated protein 9)) or Cpf1 protein (CRISPR from Prevotella and Francisella 1). A nuclease associated with the CRISPR system (for example, an endonuclease) or the like may be used. Specifically, in one embodiment, the nuclease may be a Cas protein such as Cas3, Cas9, Cpf1, Cas6, or C2c2, specifically the Cas protein of CRISPR/Cas type II, and more specifically a Cas9 protein derived from Streptococcus Pyogenes.

The term “Transcriptional Activator-Like Effector,” (TALE) as used herein, refers to DNA binding proteins comprising a TALE repeat array comprising a plurality of highly conserved 33-34 amino acid sequence comprising a highly variable two-amino acid motif (Repeat Variable Diresidue, RVD). The RVD motif determines binding specificity to a nucleic acid sequence, and can be engineered according to methods well known to those of skill in the art to specifically bind a desired DNA sequence. The simple relationship between amino acid sequence and DNA recognition has allowed for the engineering of specific DNA binding domains by selecting a combination of repeat segments containing the appropriate RVDs.

In one embodiment, the DNA binding protein may be a TALE. In yet another embodiment, the TALE may be a dual TALE module consisting of a first TALE module and a second TALE module. In some embodiments, each of the first and second TALE modules may be connected to various deaminases. For example, the first TALE module may be linked to cytosine deaminase such as DddAₜₒₓ in a full-length form, and the second TALE module may be linked to the adenine deaminase variant.

Cas9 protein is a main protein component of the CRISPR/Cas system, which can function as an activated endonuclease or nickase.

Cas9 protein or gene information thereof may be acquired from a well-known database such as the GenBank of NCBI (National Center for Biotechnology Information). For example, the Cas9 protein may be at least one selected from the group consisting of, but not limited to:
a Cas9 protein derived from Streptococcus sp., for example, Streptococcus pyogenes (e.g., SwissProt Accession number Q99ZW2 (NP_269215.1) (encoding gene: SEQ ID NO: 229);
a Cas9 protein derived from Campylobacter sp., for example, Campylobacter jejuni;
a Cas9 protein derived from Streptococcus sp., for example, Streptococcus thermophiles or Streptocuccus aureus;
a Cas9 protein derived from Neisseria meningitidis;
a Cas9 protein derived from Pasteurella sp., for example, Pasteurella multocida; and
a Cas9 protein derived from Francisella sp., for example, Francisella novicida.

Cpf1 protein, which is an endonuclease of a new CRISPR system distinguished from the CRISPR/Cas system, is small in size compared to Cas9, requires no tracrRNA, and can function with a single guide RNA. In addition, Cpf1 can recognize thymidine-rich PAM (protospacer-adjacent motif) sequences and produces cohesive double-strand breaks (cohesive end).

For example, the Cpf1 protein may be an endonuclease derived from Candidatus spp., Lachnospira spp., Butyrivibrio spp., Peregrinibacteria, Acidominococcus spp., Porphyromonas spp., Prevotella spp., Francisella spp., Candidatus Methanoplasma), or Eubacterium spp. Examples of the microorganism from which the Cpf1 protien may be derived include, but are not limited to, Parcubacteria bacterium (GWC2011_GWC2_44_17), Lachnospiraceae bacterium (MC2017), Butyrivibrio proteoclasiicus, Peregrinibacteria bacterium (GW2011_GWA_33_10), Acidaminococcus sp. (BV3L6), Porphyromonas macacae, Lachnospiraceae bacterium (ND2006), Porphyromonas crevioricanis, Prevotella disiens, Moraxella bovoculi (237), Smiihella sp. (SC_KO8D17), Leptospira inadai, Lachnospiraceae bacterium (MA2020), Francisella novicida (U112), Candidatus Methanoplasma termitum, Candidatus Paceibacter, and Eubacterium eligens.

In one embodiment, when the DNA binding protein is a Cas9 protein, the Cas9 protein may be at least one selected from the group consisting of modified Cas9 that lacks endonuclease activity and retains nickase activity as a result of introducing mutations (e.g., substitution with a different amino acid) to D10 of Streptococcus pyogenes-derived Cas9 protein (e.g., SwissProt Accession number Q99ZW2 (NP_269215.1)), and modified Cas9 protein that lacks both endonuclease activity and nickase activity as a result of introducing mutations (e.g., substitutions with different amino acids) to both D10 and H840 of Streptococcus pyogenes-derived Cas9 protein. In Cas9 protein, for example, the mutation at D10 may be D10A mutation (the amino acid D at position 10 in Cas9 protein is substituted with A), and the mutation at H840 may be H840A mutation.

If the nuclease has nickase activity, the nick may be introduced simultaneously with the diaminase-mediated base modification (e.g. cytidine converted to uradine) or sequentially, in any order, on the strand on which the base modification occurred or on the opposite strand thereof (e.g. strand opposite to the strand where base conversion occurred) (e.g., a nick is introduced at a position between the third nucleotide and the fourth nucleotide positions in the direction of the 5′ end of the PAM sequence on the opposite strand of the strand where the PAM is located). Nuclease mutations (e.g., amino acid substitutions, etc.) can occur in the catalytically active domain of the nuclease (e.g., in the case of Cas9, the RuvC catalytic domain).

In one embodiment, in the case of the Cas9 protein derived from Streptococcus Pyogenes, the mutations may be a substitution of at least one amino acid selected from the group consisting of catalytic aspartic acid at position 10 (D10), glutamic acid at position 762 (E762), histidine at position 840 (H840), asparagine at position 854 (N854), asparagine at position 863 (N863), and aspartic acid at position 986 (D986) for another amino acid. Specifically, it can include variants in which one or more amino acids selected from the group consisting of H839, H840, and N863 of Cas9 are substituted with another amino acid. Specifically, it can include variants in which amino acids at N863, H840-N863, or H839-H840-N863 of Cas9 are replaced with another amino acid. In addition to H840A, D10A SpCas9 nickase SpCas9 nickase prepared by removing some catalytic domains may also be used.

In some embodiments, the fusion protein may further include a guide RNA. The guide RNA may be, for example, at least one selected from the group consisting of CRISPR RNA (crRNA), trans-activating crRNA (tracrRNA), and single guide RNA (sgRNA). Specifically, it may be a double-stranded crRNA:tracrRNA complex in which crRNA and tracrRNA are bonded to each other, or a single-stranded guide RNA (sgRNA) in which crRNA or a portion thereof and tracrRNA or a portion thereof are linked by an oligonucleotide linker.

The adenine deaminase variant and the DNA binding protein may be used in the form of a fusion protein in which they are fused to each other directly or via a peptide linker (e.g., existing in the order of adenine deaminase variant-DNA binding protein in the N- to C-terminus direction (i.e., DNA binding protein fused to the C-terminus of adenine deaminase variant) or in the order of DNA binding protein-adenine deaminase variant in the N- to C-terminus direction (i.e., adenine deaminase variant fused to the C-terminus of DNA binding protein), a mixture of the adenine deaminase variant or mRNA coding therefor and the DNA binding protein or mRNA coding therefor, a plasmid carrying both an adenine deaminase variant-encoding gene and a DNA binding protein-encoding gene (e.g., the two genes arranged to encode the fusion protein described above, or a mixture of a adenine deaminase variant expression plasmid and a DNA binding protein expression plasmid, or a plasmid which carry an adenine deaminase variant-encoding gene and an DNA binding protein-encoding gene, respectively).

In one embodiment, the fusion protein may further comprise a cytosine deaminase. The cytosine deaminase refers to any enzyme having activity to convert a cytosine, which is found in nucleotide (e.g., cytosine present in double stranded DNA or RNA), to uracil (C-to-U conversion activity or C-to-U editing activity). The cytosine deaminase converts cytosine positioned on a strand where a PAM sequence linked to target sequence is present, to uracil. In an embodiment, the cytosine deaminase may be originated from mammals including bacteria, archaea, primates such as humans and monkeys, rodents such as rats and mice, and the like, but not be limited thereto. For example, the cytosine deaminase may be at least one selected from the group consisting of PmCDA1 (Petromyzon marinus cytosine deaminase 1) from Petromyzon marinus, DddAₜₒₓ from Burkholderia cenocepacia, and APOBEC (apolipoprotein B mRNA editing enzyme, catalytic polypeptide-like) family, but not be limited thereto.

In some embodiments, the cytosine deaminase is wild-type Petromyzon marinus CDA1 (pmCDA1) or a catalytic domain thereof. In some embodiments, the cytosine deaminase comprises one or more mutations in the pmCDA1 sequence, such that the editing efficiency, and/or substrate editing preference of pmCDA1 is changed according to specific needs.

pmCDA1 has the following amino acid sequence:

(SEQ ID NO: 2)
MTDAEYVRIHEKLDIYTFKKOFFNNKKSVSHRCYVLFELKRRGER
RACFWGYAVNKPQSGTERGIHAEIFSIRKVEEYLRDNPGQFTINW
YSSWSPCADCAEKILEWYNQELRGNGHTLKIWACKLYYEKNARNQ
IGLWNLRDNGVGLNVMVSEHYQCCRKIFIQSSHNQLNENRWLEKT
LKRAEKRRSELSIMIQVKILHTTKSPAVSRGSG

In some embodiments, as an example of a deaminase, DddA tox is cytotoxic, and thus, in order to avoid toxicity in host cells, DddAₜₒₓ is split into two inactive halves, each of which is fused to a DNA-binding protein in a DddA-derived cytosine base editor (DdCBE). A functional deaminase is reassembled at a target DNA site, when two inactive halves are brought together by the DNA-binding protein. The full-length DddA tox has the following amino acid sequence:

GSYALGPYQISAPOLPAYNGQTVGTFYYVNDAGGLESKVFSSGGPTPYPNYANA GHVEGQSALFMRDNGISEGLVFHNNPEGTCGFCVNMTETLLPENAKMTVVPPEGAIPVK RGATGETKVFTGNSNSPKSPTKGGC (SEQ ID NO: 3) which corresponds to PDB Accession No. 6U08 A of Bu rkhol.de ria cenocepacia), and can include fragments or variants thereof, including amino acid sequences having at least 70%, at least 75%, at least 80%, at least 85%, at least 90%, at least 95%, at least 96%, at least 97%, at least 98%, at least 99%, or 100% sequence identify with DddA of 6U08_A.

In another embodiment, the APOBEC (apolipoprotein B mRNA editing enzyme, catalytic polypeptide-like) family, for example, may be at least one selected from the following group, but not be limited to:
APOBEC1: Homo sapiens APOBEC1 (Protein: GenBank Accession Nos. NP_001291495.1, NP_001635.2, NP_005880.2, etc.; gene (mRNA or cDNA; described in the order of the above listed corresponding proteins): GenBank Accession Nos. NM_001304566.1, NM_001644.4, NM_005889.3, etc.), Mus musculus APOBEC1 (protein: GenBank Accession Nos. NP_001127863.1, NP_112436.1, etc.; gene: GenBank Accession Nos. NM_001134391.1, NM_031159.3, etc.);
APOBEC2: Homo sapiens APOBEC2 (protein: GenBank Accession No. NP_006780.1, etc.; gene: GenBank Accession No. NM_006789.3 etc.), mouse APOBEC2 (protein: GenBank Accession No. NP_033824.1, etc.; gene: GenBank Accession No. NM_009694. 3, etc.);
APOBEC3B: Homo sapiens APOBEC3B (protein: GenBank Accession Nos. NP_001257340.1, NP_004891.4, etc.; gene: GenBank Accession Nos. NM_001270411.1, NM_004900.4, etc.), Mus musculus APOBEC3B (proteins: GenBank Accession Nos. NP_001153887.1, NP_001333970.1, NP_084531.1, etc.; gene: GenBank Accession Nos. NM_001160415.1, NM_001347041.1, NM_030255.3, etc.);
APOBEC3C: Homo sapiens APOBEC3C (protein: GenBank Accession No. NP_055323.2 etc.; gene: GenBank Accession No. NM_014508.2 etc.);
APOBEC3D (including APOBEC3E): Homo sapiens APOBEC3D (protein: GenBank Accession No. NP_689639.2, etc.; gene: GenBank Accession No. NM_152426.3 etc.);
APOBEC3F: Homo sapiens APOBEC3F (protein: GenBank Accession Nos. NP_660341.2, NP_001006667.1, etc.; gene: GenBank Accession Nos. NM_145298.5, NM_001006666.1, etc.);
APOBEC3G: Homo sapiens APOBEC3G (protein: GenBank Accession Nos. NP_068594.1, NP_001336365.1, NP_001336366.1, NP_001336367.1, etc.; gene: GenBank Accession Nos. NM_021822.3, NM_001349436.1, NM_001349437.1, NM_001349438.1, etc.);
APOBEC3H: Homo sapiens APOBEC3H (protein: GenBank Accession Nos. NP_001159474.2, NP_001159475.2, NP_001159476.2, NP_861438.3, etc.; gene: GenBank Accession Nos. NM_001166002.2, NM_001166003. 2, NM_001166004.2, NM_181773.4, etc.);
APOBEC4 (including APOBEC3E): Homo sapiens APOBEC4 (protein: GenBank Accession No. NP_982279.1, etc.; gene: GenBank Accession No. NM_203454.2 etc.); mouse APOBEC4 (protein: GenBank Accession No. NP_001074666.1, etc.; gene: GenBank Accession No. NM_001081197.1, etc.); and
Activation-induced cytidine deaminase (AICDA or AID): Homo sapiens AID (Protein: GenBank Accession Nos. NP_001317272.1, NP_065712.1, etc; Genes: GenBank Accession Nos. NM_001330343.1, NM_020661.3, etc.); mouse AID (protein: GenBank Accession No. NP_033775.1, etc., gene: GenBank Accession No. NM_009645.2, etc.), and the like.

The cytosine deaminase may be a non-toxic full-length deaminase (i.e. monomeric cytosine deaminase) or be in a two-split form (i.e. dimeric deaminase) comprising separated first and second domains, each of which may be characterized by the absence of deaminase activity.

In another embodiment, the adenine deaminase variant may bind to the N- or C-terminus of a DNA binding protein or cytosine deaminase or variant thereof. For example, in case the DNA-binding protein is ZFP, the adenine deaminase variant is TadA8e variant, and the cytosine deaminase or its variant is DddAₜₒₓ, they can be included in the following order, but are not limited thereto: ZFP-TadA8e variant-DddAₜₒₓ, ZFP-DddAₜₒₓ-TadA8e variant, TadA-DddAₜₒₓ-ZFP, or DddAtox-TadA8e variant-ZFP.

In some embodiments, when the cytosine deaminase is contained in a split form, and the DNA binding protein is a zinc finger protein, the C-terminus of the first domain of the cytosine deaminase is bound to the N-terminus of the zinc finger protein (ZF-Left), and the N-terminus of the second domain of the cytosine deaminase is bound to the C-terminus of a zinc finger protein (ZF-Right) (NC configuration), the adenine deaminase variant may be attached to the C-terminus of the zinc finger protein (ZF-Left), the N-terminus or C-terminus of the first domain of the cytosine deaminase, the N-terminus of zinc finger protein (ZF-Right), or the N-terminus or C-terminus of the second domain of the cytosine deaminase. In various embodiments, the adenine deaminase variant may bind to:
the C-terminus of the zinc finger protein (ZF-Left) attached to the N-terminus of the first domain of cytosine deaminase, and C-terminus of the zinc finger protein (ZF-Right) attached to the N-terminus of the second domain of cytosine deaminase. (CC configuration);
the C-terminus of the zinc finger protein (ZF-Left) attached to the N-terminus of the first domain of cytosine deaminase, and the N-terminus of the zinc finger (ZF-Right) attached to the C-terminus of the second domain of cytosine deaminase (CN configuration); or
the N-terminus of the zinc finger protein (ZF-Left) attached to the C-terminus of the first domain of cytosine deaminase, and the N-terminus of zinc finger protein (ZF-Right) attached to the C-terminus of the second domain of cytosine deaminase. (NN configuration).

Thus, in some embodiments, the adenine deaminase variant may bind to C-terminus of zinc finger protein (ZF-Left), N-terminus or C-terminus of the first domain of the cytosine deaminase, zinc finger protein (ZF-Right), or N-terminal or C-terminal of the second domain of the cytosine deaminase.

In some embodiments, when the cytosine deaminase is in a split form and the DNA binding protein is a TALE, the first domain of the cytosine deaminase is attached to a first TALE, the second domain of the cytosine deaminase is attached to a second TALE, and each has a structure of N′-TALE-first domain DDDA-C′ and N′-TALE-second domain DDDA-C′, respectively. The adenine deaminase variant may bind to the N-terminus or C-terminus of the first domain of cytosine deaminase or the N-terminus or C-terminus of the second domain of cytosine deaminase.

In one embodiment, when the cytosine deaminase is included in a full-length form and the DNA binding protein is a TALE, it can include a single TALE module, including a single TALE module and a cytosine deaminase in the NC orientation, wherein an adenine deaminase variant can bind to the C-terminus of the single TALE module, or to the N- or C-terminus of the cytosine deaminase.

In yet another embodiment, when the cytosine deaminase is included in a full-length form and the DNA binding protein is a TALE, a dual TALE module may be included. A first TALE module and cytosine deaminase are included in the N—C direction, and a second domain including an adenine deaminase variant and a second TALE may be further included. With the structure of N′-TALE-cytosine deaminase-C′ and N′-TALE-adenine deaminase variant-C′, the adenine deaminase variant can bind to the N-terminus or C-terminus of TALE.

In one embodiment, the fusion protein may further comprise UGI (uracil glycosylase inhibitor). UGI can increase the efficiency of base correction by inhibiting the activity of UDG (Uracil DNA glycosylase), an enzyme that repairs mutant DNA that catalyzes the removal of U from DNA.

In another embodiment, the DNA may be nuclear DNA or organellar DNA.

In one embodiment, the fusion protein may further comprise NLS (nuclear localization signal). The nuclear localization signal protein may be, for example, derived from the simian virus 40 large tumor antigen (SV40 large T antigen), but is not limited thereto. The nuclear localization signal protein may contain, for example, the following amino acid sequence, but is not limited thereto:

(SEQ ID NO: 4)
PKKKRKV

In another embodiment, the fusion protein may further comprise MTS (mitochondrial targeting sequence) or CTP (chloroplast transit peptide). The mitochondrial targeting sequence protein may be, for example, SOD2-MTS or COX8A-MTS, and may contain the following amino acid sequences, but are not limited thereto:

SOD2-MTS:
(SEQ ID NO: 5)
LSRAVCGTSRQLAPVLGYLGSRQKHSLPD
COX8A-MTS:
(SEQ ID NO: 6)
SVLTPLLLRGLTGSARRLPVPRAKIHSL.

The chloroplast transit peptide protein may be, for example, derived from Arabidopsis RECA1 but is not limited thereto. The chloroplast transit peptide protein may contain, for example, the following amino acid sequence, but is not limited thereto:

(SEQ ID NO: 7)
MDSQLVLSLKLNPSFTPLSPLFPFTPCSSFSPSLRFSSC
YSRRLYSPVTVYAAK.

In another embodiment, the fusion protein may further comprise NES (nuclear export signal). The nuclear export signal protein may be, for example, derived from MVM (Mirute virus of mice), but is not limited thereto. The nuclear export signal protein may contain, for example, the following amino acid sequence, but is not limited thereto:

(SEQ ID NO: 8)
VDEMTKKFGTLTIHDTEK

In one embodiment, when the signal peptides are attached to the fusion protein, the structure may be as follows: Signal peptide-DNA binding protein-Deaminase. In another embodiment, the structure could be Signal Peptide-Deaminase-DNA binding protein. In one embodiment, the nuclear export signal protein, CTP (chloroplast transit peptide), or a polynucleotide encoding the same may be attached to the N-terminus of a DNA-binding protein, cytosine deaminase (DdCBE), or a polynucleotide encoding the same.

In various embodiments, the fusion protein may further comprise a nickase, for example, MutH, MutH variants, or Nt.BspD6I(C), but not limited thereto. MutH is a weak endonuclease that is activated once bound to MutL. It nicks unmethylated DNA and the unmethylated strand of hemimethylated DNA but does not nick fully methylated DNA. On the other hand, nicking endonuclease Nt.BspD6I (Nt.BspD6I) is the large subunit of the heterodimeric restriction endonuclease R.BspD6I. It recognizes the short specific DNA sequence 5″-GAGTC and cleaves only the top strand in dsDNA at a distance of four nucleotides downstream the recognition site toward the 3″-terminus. The resulting strand-specific nick leads to the production of transient single-stranded DNA. Since TadA variants are nucleobase deaminases that specifically target single-stranded DNA, they have the ability to induce A-to-G editing in close proximity to the nick site. In another embodiment, the fusion protein further comprising a nickase may be in a dimeric form comprising a first fusion protein and a second protein. The first fusion protein may include the DNA-binding protein, and the adenine deaminase variant, and the second protein may include another DNA-binding protein, and the nickase.

In one embodiment, a polynucleotide encoding the adenine deaminase or the fusion protein is provided. The term “polynucleotide” is used interchangeably with “nucleic acid”, “oligonucleotide”, “nucleotide”, “nucleotide sequence.” It can contain polymeric forms of nucleotides of any length, deoxyribonucleotides or ribonucleotides, or analogs thereof. Polynucleotides can have any three-dimensional structure and can perform any known or unknown function. A polynucleotide can comprise one or more modified nucleotides, such as methylated nucleotides and nucleotide analogs. Modifications to the nucleotide structure are possible before or after assembly of the polymer.

The nucleic acid can be an RNA sequence, in particular an mRNA sequence, a DNA sequence, or a combination thereof (RNA-DNA combination sequences).

The nucleic acid may be delivered using a viral vector such as Adeno-Associated Viral Vector (AAV), Adenoviral Vector (AdV), Lentiviral Vector (LV) Retroviral Vector (RV), or other viral vectors such as episomal vector including Simian virus 40 (SV40) ori, bovine papilloma virus (BPV) ori or Epstein-Barr nuclear antigen (EBV). In various embodiment, the delivery may be also carried out using a non-viral vector, or through plasmid or mRNA delivery.

The vector may be delivered in vivo or into cells by a local injection method (e.g., direct injection into a lesion or target site), electroporation, lipofection, viral vector, nanoparticles, PTD (protein translocation domain) fusion protein method, or the like.

As means for expressing the above proteins, known expression vectors such as plasmid vectors, cosmid vectors and bacteriophage vectors can be used. Such vectors can be readily prepared by those skilled in the art according to any known method using DNA recombination techniques.

A recombinant expression vector is designed to carry nucleic acid in a format that facilitates its expression within a host cell. The nucleic acid sequence intended for expression is operably linked to the recombinant expression vector, and this vector is equipped with one or more regulatory elements, which can be chosen according to the specific host cell. In a recombinant expression vector, “operably linked” means that the nucleotide sequence of interest is linked to a regulatory element in a manner that allows expression of the nucleotide sequence. (e.g., in an in vitro transcription/translation system or in a host cell when the vector is introduced to the host cell).

In one embodiment, provided is a base editing composition for A-to-G base editing in DNA comprising the fusion protein, a polynucleotide encoding the fusion protein or an expression vector comprising the polynucleotide. The fusion protein has been explained above in detail.

In one embodiment, the fusion protein of the base editing composition may further comprise a cytosine deaminase. The cytosine deaminase has been explained above. In some embodiments, the cytosine deaminase may be present in a two split form, and the fusion protein may comprise a first fusion protein comprising a first split of the cytosine deaminase and a second fusion protein comprising a second split of the cytosine deaminase. The first split may comprise the amino acid sequence of SEQ ID NO: 9 or 10, and the second split may comprise the amino acid sequence of SEQ ID NO: 11 or 12, but is not limited thereto. As an embodiment, for example, two TALEDs may consist of the left- or right-side TALE fused to the N-terminal DddAₜₒₓ half split at G1397 (L-1397N or R-1397N, respectively) and the right- or left-side TALE fused to the C-terminal DddAₜₒₓ half split at G1397 and TadA8e (R-1397C-AD or L-1397C-AD).

| | |
|---|---|
| 1333 | PTPYPNYANAGHVEGQSALFMRDNGISEGLVFHNNPEGTCGFCVNMTETL |
| DddAₜₒₓ | LPENAKMTVVPPEGAIPVKRGATGETKVFTGNSNSPKSPTKGGC (SEQ ID |
| -C- | NO: 9), and can include fragments or |
| | variants thereof, including amino acid |
| | sequences having at least 70%, at least |
| | 75%, at least 80%, at least 85%, at least 90%, |
| | at least 95%, at least 96%, at least 97%, |
| | at least 98%, at least 99%, or 100% sequence |
| | identify with DddA of SEQ ID NO: 9. |
| 1333 | GSYALGPYQISAPQLPAYNGQTVGTFYYVNDAGGLESKVFSSGG (SEQ ID |
| DddAₜₒₓ | NO: 10) and can include fragments or variants |
| -N- | thereof, including amino acid |
| | sequences having at least 70%, at least 75%, |
| | at least 80%, at least 85%, at least 90%, |
| | at least 95%, at least 96%, at least 97%, at |
| | least 98%, at least 99%, or 100% sequence |
| | identify with DddA of SEQ ID NO: 10. |
| 1397 | AIPVKRGATGETKVFTGNSNSPKSPTKGGC |
| DddAₜₒₓ | (SEQ ID NO: 11), and can include |
| -C- | fragments or variants thereof, including |
| | amino acid sequences having at least 70%, |
| | at least 75%, at least 80%, at least 85%, |
| | at least 90%, at least 95%, at least 96%, at |
| | least 97%, at least 98%, at least 99%, or |
| | 100% sequence identify with DddA of SEQ |
| | ID NO: 11. |
| 1397 | GSYALGPYQISAPQLPAYNGQTVGTFYYVNDAGGLESKVFSSGGPTPYPN |
| DddAₜₒₓ | YANAGHVEGQSALFMRDNGISEGLVFHNNPEGTCGFCVNMTETLLPENA |
| -N- | KMTVVPPEG (SEQ ID NO: 12), and can include |
| | fragments or variants thereof, |
| | including amino acid sequences having at |
| | least 70%, at least 75%, at least 80%, at |
| | least 85%, at least 90%, at least 95%, at |
| | least 96%, at least 97%, at least 98%, at |
| | least 99%, or 100% sequence identify with |
| | DddA of SEQ ID NO: 12. |

In various embodiments, the base editing composition may exhibits remarkably reduced off-target effects involving an unintended base alteration in DNA and/or RNA. In another embodiment, the base editing composition may reduce unwanted bystander effects while narrowing activity windows. Alternatively, the base editing composition may induce base editing only at a single nucleotide residue without any intended off-target editing in the target DNA with a frequency of at least 10%, at least 11%, at least 12%, at least 13%, at least 14%, or at least 15%.

Yet in another embodiment, provided is a method for A-to-G base editing in DNA comprising delivering the base editing composition to a cell containing a target DNA.

The cell may be eukaryotic cells (e.g., fungi such as yeast, eukaryotic animals and/or eukaryotic plant-derived cells (e.g., embryonic cells, stem cells, somatic cells, gametes, etc.), eukaryotic animals (e.g., humans, monkeys, primates dogs, pigs, cattle, sheep, goats, mice, rats, etc.), or eukaryotic plants (e.g., algae such as green algae, corn, soybeans, wheat, rice, etc.), but is not limited thereto.

The delivery of the base editing composition to a cell containing a target DNA may be carried out ex vivo or in vivo.

In some embodiments, the target DNA may be nuclear DNA, organellar DNA, or mitochondrial DNA of a human subject with a hereditary disease.

As used herein, the term “hereditary disease” refers to a pathological condition that occurs due to a mutation that is harmful to a gene or chromosome. Examples of the hereditary diseases include, but are not limited to, mitochondrial encephalopathy, lactic acidosis, and stroke-like episodes (MELAS) syndrome, DEAF, leber hereditary optic neuropathy (LHON), leigh Syndrome, Myopath and Chronic progressive external ophthalmoplegia (CPEO)

The method may method may exhibit reduced off-target effects compared to a case when using a base editor comprising an adenine deaminase having the amino acid sequence of SEQ ID NO: 1, wherein the off-target editing is characterized by an unintended base alteration in DNA and/or RNA. In another embodiment, the method may reduce unwanted bystander effects while narrowing activity windows compared to when using a base editor comprising an adenine deaminase having amino acids represented by SEQ ID: 1 mutation. Alternatively, the method may induce base editing only at a single nucleotide residue without any intended off-target editing in the target DNA with a frequency of at least 10%, at least 11%, at least 12%, at least 13%, at least 14%, or at least 15%.

In another embodiment, the method may exhibit a reduced off-target effect compared to when using a base editor comprising an adenine deaminase having amino acids represented by SEQ ID: 1 with V106W mutation, wherein the off-target editing is characterized by an unintended base alteration in DNA and/or RNA. In another embodiment, the method may reduce unwanted bystander effects while narrowing activity windows compared to when using a base editor comprising an adenine deaminase having amino acids represented by SEQ ID: 1 with V106W mutation. Alternatively, the method may induce base editing only at a single nucleotide residue without any intended off-target editing in the target DNA with a frequency of at least 10%, at least 11%, at least 12%, at least 13%, at least 14%, or at least 15%.

In one embodiment, provided is a method for reducing off-target effect and/or unwanted bystander effects while narrowing activity windows, the method comprising delivering the base editing composition to a cell containing a target DNA.

The cell may be eukaryotic cells (e.g., fungi such as yeast, eukaryotic animals and/or eukaryotic plant-derived cells (e.g., embryonic cells, stem cells, somatic cells, gametes, etc.), eukaryotic animals (e.g., humans, monkeys, primates dogs, pigs, cattle, sheep, goats, mice, rats, etc.), or eukaryotic plants (e.g., algae such as green algae, corn, soybeans, wheat, rice, etc.), but is not limited thereto.

In some embodiments, the target DNA may be nuclear DNA, organellar DNA, or mitochondrial DNA of a human subject with a hereditary disease. The hereditary disease has been defined above. The method may method may exhibit reduced off-target effects compared to a case when using a base editor comprising an adenine deaminase having the amino acid sequence of SEQ ID NO:1, wherein the off-target editing is characterized by an unintended base alteration in DNA and/or RNA. In another embodiment, the method may reduce unwanted bystander effects while narrowing activity windows compared to when using a base editor comprising an adenine deaminase having amino acids represented by SEQ ID: 1. Alternatively, the method may induce base editing only at a single nucleotide residue without any intended off-target editing in the target DNA with a frequency of at least 10%, at least 11%, at least 12%, at least 13%, at least 14%, or at least 15%.

In another embodiment, the method may exhibit a reduced off-target effect compared to when using a base editor comprising an adenine deaminase having amino acids represented by SEQ ID: 1 with V106W mutation, wherein the off-target editing is characterized by an unintended base alteration in DNA and/or RNA. In another embodiment, the method may reduce unwanted bystander effects while narrowing activity windows compared to when using a base editor comprising an adenine deaminase having amino acids represented by SEQ ID: 1 with V106W mutation. Alternatively, the method may induce base editing only at a single nucleotide residue without any unintended off-target editing in the target DNA with a frequency of at least 10%, at least 11%, at least 12%, at least 13%, at least 14%, or at least 15%.

Yet, in another embodiment, provided is a use of the adenine deaminase variant or the fusion protein in A-to-G base editing in DNA and/or reducing off-target effect in A-to-G base editing in DNA, or in preparing a composition for A-to-G base editing in DNA and/or reducing off-target effect in A-to-G base editing in DNA. The adenine deaminase variant and the fusion protein have been explained above in detail.

In one embodiment, the fusion protein may further comprise a cytosine deaminase. The cytosine deaminase has been explained above. In some embodiments, the cytosine deaminase may be present in two split form, and the fusion protein comprises a first fusion protein comprising a first split of the cytosine deaminase and a second fusion protein comprising a second split of the cytosine deaminase. The first split may comprise the amino acid sequence of SEQ ID NO: 9 or 10 and the second split may comprise the amino acid sequence of SEQ ID NO: 11 or 12, but is not limited thereto.

In various embodiments, the composition for A-to-G base editing in DNA and/or reducing off-target effect in A-to-G base editing in DNA may exhibit remarkably reduced off-target effects involving an unintended base alteration in DNA and/or RNA. In one embodiment, the composition may reduce unwanted bystander effects while narrowing activity windows Alternatively, the composition may induce base editing only at a single nucleotide residue without any unintended off-target editing in the target DNA with a frequency of at least 10%, at least 11%, at least 12%, at least 13%, at least 14%, or at least 15%.

Hereinafter, the present invention will be described in more detail below with reference to examples. It will be apparent to those skilled in the art that these examples are intended solely to illustrate the invention and that the scope of the invention is not to be construed as being limited by these examples.

### REFERENCE EXAMPLES

### 1. Preparation of Cell Lines

HEK 293T cells were purchased from the American Type Culture Collection (ATCC) (CRL-11268). NIH3T3 and B16F10 cells were purchased from ATCC (CRL-1658, CRL-6475). HEK 293T cells were cultured in Dulbecco's Modified Eagle Medium (DMEM; Welgene) supplemented with 10% (v/v) fetal bovine serum (Welgene) and 1% (v/v) antibiotic-antimycotic solution (Welgene). NIH3T3 and B16F10 cells were cultured in DMEM supplemented with 10% (v/v) bovine calf serum (Gibco) for NIH3T3 cells or 10% fetal bovine serum (Gibco) for B16F10 cells in the absence of any antibiotics. The cells were incubated at 37° C. with 5% CO₂. All cell lines were passaged before approaching 90% confluency.

### 2. PyMOL Analysis

The ABE8e (PDB accession number 6VPC) structure was downloaded from PDB and visualized with PyMOL v.2.5.4. Some elements including Cas9, single guide RNA, and double-stranded DNA were excluded from the PDB file; 8AZ, a transition state analog for adenosine deamination reactions, and the TadA monomer were retained. 11 residues (V28, V30, N46, A48, 149, V82, F84, V106, N108, K110, and R111) that were near 8AZ, including residues previously known to contact DNA, were selected.

### 3. Plasmid Construction

To construct plasmids encoding DdCBEs, DddA-split TALEDs (sTALEDs), and mTALEDs that target specific sites, plasmids containing a stuffer, which is a sequence between two restriction enzyme sites that is helpful when separating fragments during gel electrophoresis were prepared. The architectures of the plasmids are as follows: STALED plasmids (p3s-stuffer-DddAₜₒₓ half (1397C)-AD and p3s-stuffer-DddAₜₒₓ half (1397N)); mTALED plasmids (p3s-stuffer-E1347A DddAₜₒₓ full-AD). These plasmids were obtained from Addgene (DdCBE, #187168, #187171, #187173, #178174; sTALED, #187167, #187169, #187170, #187172; mTALED, #187163, #187166). Plasmids encoding DdCBEs, sTALEDs, and mTALEDs targeting specific sites were constructed by inserting custom-designed TALE array sequences as shown in Table 1 below. To remove the stuffer sequences, each plasmid digested with BsaI or BsmbI (NEB), and inserts, including a custom-designed TALE array sequence synthesized by IDT, were inserted into the digested vector using a HiFi DNA assembly kit (NEB). Alternatively, the desired TALED construct was generated by digesting the master vector with BsaI or BsmbI to cleave the site in the stuffer and then assembling six TALE arrays in that position using the Golden Gate method.

TABLE 1

| | Left-mitoTALE amino | Right-mitoTALE amino |
|---|---|---|
| Site | acid sequence | acid sequence |
| COX | LTPDQVVAIASHDGGKQALETVQRLLP | LTPDQVVAIASNIGGKQALETVQRLLP |
| 3.1 | VLCQAHGLTPAQVVAIASHDGGKQAL | VLCQDHGLTPAQVVAIASNNGGKQAL |
| | ETVQRLLPVLCQDHGLTPAQVVAIASN | ETVQRLLPVLCQDHGLTPAQVVAIASN |
| | GGGKQALETVQRLLPVLCQDHGLTPD | IGGKQALETVQRLLPVLCQAHGLTPAQ |
| | QVVAIASNIGGKQALETVQRLLPVLCQ | VVAIASNGGGKQALETVQRLLPVLCQ |
| | DHGLTPDQVVAIASHDGGKQALETVQ | AHGLTPDQVVAIASNNGGKQALETVQ |
| | RLLPVLCQDHGLTPAQVVAIASNIGGK | RLLPVLCQAHGLTPAQVVAIASHDGG |
| | QALETVQRLLPVLCQAHGLTPAQVVAI | KQALETVQRLLPVLCQAHGLTPDQVV |
| | ASNIGGKQALETVQRLLPVLCQDHGLT | AIASHDGGKQALETVQRLLPVLCQDH |
| | PDQVVAIASNNGGKQALETVQRLLPVL | GLTPDQVVAIASNNGGKQALETVQRL |
| | CQDHGLTPEQVVAIASHDGGKQALET | LPVLCQDHGLTPAQVVAIASNGGGKQ |
| | VQRLLPVLCQAHGLTPAQVVAIASHD | ALETVQRLLPVLCQAHGLTPEQVVAIA |
| | GGKQALETVQRLLPVLCQDHGLTPAQ | SHDGGKQALETVQRLLPVLCQAHGLT |
| | VVAIASNGGGKQALETVQRLLPVLCQ | PAQVVAIASNNGGKQALETVQRLLPVL |
| | DHGLTPAQVVAIASHDGGKQALETVQ | CQDHGLTPDQVVAIASNNGGKQALET |
| | RLLPVLCQAHGLTPDQVVAIASNIGGK | VQRLLPVLCQDHGLTPDQVVAIASNIG |
| | QALETVQRLLPVLCQDHGLTPAQVVAI | GKQALETVQRLLPVLCQAHGLTPAQV |
| | ASNNGGKQALETVQRLLPVLCQDHGL | VAIASNIGGKQALETVQRLLPVLCQDH |
| | TPDQVVAIASNIGGKQALETVQRLLPV | GLTPAQVVAIASNIGGKQALETVQRLL |
| | LCQDHGLTPAQVVAIASNNGGKQALE | PVLCQAHGLTPEQVVAIASNGGGKQA |
| | TVQRLLPVLCQDHGLTPEQVVAIASNG | LETVQRLLPVLCQAHGLTPAQVVAIAS |
| | GGKQALE | NNGGKQALETVQRLLPVLCQDHGLTP |
| | (SEQ ID NO: 13) | DQVVAIASNNGGKQALETVQRLLPVL |
| | | CQDHGLTPEQVVAIASNGGGKQALE |
| | | (SEQ ID NO: 14) |
| ND1 | LTPDQVVAIASHDGGKQALETVQRLLP | LTPDQVVAIASNNGGKQALETVQRLLP |
| | VLCQAHGLTPDQVVAIASNGGGKQAL | VLCQDHGLTPAQVVAIASNIGGKQALE |
| | ETVQRLLPVLCQDHGLTPAQVVAIASN | TVQRLLPVLCQAHGLTPEQVVAIASNN |
| | IGGKQALETVQRLLPVLCQDHGLTPDQ | GGKQALETVQRLLPVLCQAHGLTPDQ |
| | VVAIASNNGGKQALETVQRLLPVLCQ | VVAIASNGGGKQALETVQRLLPVLCQ |
| | AHGLTPDQVVAIASHDGGKQALETVQ | AHGLTPAQVVAIASNGGGKQALETVQ |
| | RLLPVLCQDHGLTPDQVVAIASHDGG | RLLPVLCQAHGLTPEQVVAIASNGGGK |
| | KQALETVQRLLPVLCQAHGLTPAQVV | QALETVQRLLPVLCQAHGLTPAQVVAI |
| | AIASNGGGKQALETVQRLLPVLCQDH | ASNNGGKQALETVQRLLPVLCQDHGL |
| | GLTPAQVVAIASNIGGKQALETVQRLL | TPAQVVAIASNIGGKQALETVQRLLPV |
| | PVLCQAHGLTPDQVVAIASNNGGKQA | LCQAHGLTPEQVVAIASNGGGKQALE |
| | LETVQRLLPVLCQAHGLTPAQVVAIAS | TVQRLLPVLCQAHGLTPDQVVAIASNN |
| | HDGGKQALETVQRLLPVLCQDHGLTP | GGKQALETVQRLLPVLCQAHGLTPAQ |
| | DQVVAIASHDGGKQALETVQRLLPVL | VVAIASHDGGKQALETVQRLLPVLCQ |
| | CQAHGLTPEQVVAIASNNGGKQALET | DHGLTPDQVVAIASNGGGKQALETVQ |
| | VQRLLPVLCQAHGLTPAQVVAIASNG | RLLPVLCQAHGLTPDQVVAIASHDGG |
| | GGKQALETVQRLLPVLCQAHGLTPAQ | KQALETVQRLLPVLCQAHGLTPDQVV |
| | VVAIASNGGGKQALETVQRLLPVLCQ | AIASNIGGKQALETVQRLLPVLCQAHG |
| | AHGLTPEQVVAIASNGGGKQALE | LTPAQVVAIASHDGGKQALETVQRLLP |
| | (SEQ ID NO: 15) | VLCQAHGLTPAQVVAIASHDGGKQAL |
| | | ETVQRLLPVLCQDHGLTPAQVVAIASH |
| | | DGGKQALETVQRLLPVLCQAHGLTPE |
| | | QVVAIASNGGGKQALE |
| | | (SEQ ID NO: 16) |
| ND6 | LTPAQVVAIASHDGGKQALETVQRLLP | LTPEQVVAIASNIGGKQALETVQRLLP |
| | VLCQDHGLTPEQVVAIASNIGGKQALE | VLCQAHGLTPDQVVAIASHDGGKQAL |
| | TVQRLLPVLCQAHGLTPAQVVAIASNI | ETVQRLLPVLCQAHGLTPDQVVAIASN |
| | GGKQALETVQRLLPVLCQDHGLTPAQ | GGGKQALETVQRLLPVLCQDHGLTPD |
| | VVAIASHDGGKQALETVQRLLPVLCQ | QVVAIASNIGGKQALETVQRLLPVLCQ |
| | AHGLTPDQVVAIASHDGGKQALETVQ | DHGLTPDQVVAIASHDGGKQALETVQ |
| | RLLPVLCQDHGLTPDQVVAIASHDGG | RLLPVLCQDHGLTPAQVVAIASNIGGK |
| | KQALETVQRLLPVLCQDHGLTPAQVV | QALETVQRLLPVLCQAHGLTPAQVVAI |
| | AIASHDGGKQALETVQRLLPVLCQAH | ASNNGGKQALETVQRLLPVLCQAHGL |
| | GLTPAQVVAIASNGGGKQALETVQRL | TPDQVVAIASHDGGKQALETVQRLLPV |
| | LPVLCQDHGLTPDQVVAIASNNGGKQ | LCQDHGLTPAQVVAIASNNGGKQALE |
| | ALETVQRLLPVLCQAHGLTPEQVVAIA | TVQRLLPVLCQAHGLTPEQVVAIASNI |
| | SNIGGKQALETVQRLLPVLCQAHGLTP | GGKQALETVQRLLPVLCQAHGLTPAQ |
| | AQVVAIASHDGGKQALETVQRLLPVL | VVAIASNGGGKQALETVQRLLPVLCQ |
| | CQAHGLTPAQVVAIASHDGGKQALET | AHGLTPAQVVAIASNNGGKQALETVQ |
| | VQRLLPVLCQDHGLTPAQVVAIASHD | RLLPVLCQDHGLTPDQVVAIASNNGG |
| | GGKQALETVQRLLPVLCQDHGLTPAQ | KQALETVQRLLPVLCQAHGLTPAQVV |
| | VVAIASHDGGKQALETVQRLLPVLCQ | AIASHDGGKQALETVQRLLPVLCQDH |
| | AHGLTPAQVVAIASHDGGKQALETVQ | GLTPDQVVAIASNGGGKQALETVQRL |
| | RLLPVLCQDHGLTPDQVVAIASNIGGK | LPVLCQAHGLTPEQVVAIASNIGGKQA |
| | QALETVQRLLPVLCQAHGLTPEQVVAI | LETVQRLLPVLCQAHGLTPDQVVAIAS |
| | ASNGGGKQALE | NGGGKQALETVQRLLPVLCQDHGLTP |
| | (SEQ ID NO: 17) | EQVVAIASNGGGKQALE |
| | | (SEQ ID NO: 18) |

### 4. Cell Culture and Transfection

HEK 293T cells were grown in DMEM (Welgene) with 10% fetal bovine serum (Welgene) and 1% antibiotic-antimycotic solution (Welgene). NIH3T3 (CRL-1658, ATCC) and B16F10 (CRL-6475, ATCC) cells were grown in DMEM supplemented with 10% (v/v) bovine calf serum (Gibco) for NIH3T3 cells or 10% fetal bovine serum (Gibco) for B16F10 cells in the absence of any antibiotics. Cell lines were maintained at 37° C. with 5% CO₂ and were passaged before reaching 90% confluency, with timing depending on the doubling period of the specific cell line.

For HEK 293T cells, cells were seeded onto 48-well plates (Corning) at a density of 7.5×10⁴ cells per well prior to transfection. The cells were transfected with plasmids (total 1 ug) using 1.5 μL of Lipofectamine 2000 (Invitrogen) after 24 h. For delivery of sTALED pair, DdCBE pair, and CRISPR-Base Editor with sgRNA, the total amount of plasmid was 1 μg (500 ng each). When single construct was used, the amount of transfected plasmid was 500 ng. After 96 h, the transfected cells were harvested. For NIH3T3 and B16F10 cells, cells were seeded in 24-well cell culture plates (SPL, Seoul, Korea) at a density of 1×10⁵ cells per well, 18-24 h before transfection. Lipofection using Lipofectamine 3000 (Invitrogen) was performed with 500 ng of each sTALED-encoding plasmid to make up 1000 ng of total plasmid DNA. For mTALED, 500 ng of plasmid was used. Cells were harvested 3 days after transfection.

### 5. Transcriptome Sequencing

Total RNA was isolated using a NucleoSpin RNA Kit (MN #Macherey-Nagel) according to the manufacturer's instructions at 48 h or 96 h post-transfection. RNA libraries were prepared using a TruSeq Stranded Total RNA Library Prep Gold kit (Illumina). RNA library quality was assessed using a 2200 TapeStation with a D1000 ScreenTape system (Agilent). Total RNA sequencing was performed using a NovaSeq 6000 Sequencer (Illumina) at Macrogen with paired-end sequencing systems (2×10⁰ bp).

### 6. RNA Variant Calling

To analyze NGS data from RNA sequencing, a reference to a RNA variant-calling pipeline previously used for RNA off-target analysis of CRISPR DNA base editors was made. Briefly, the fastq sequencing reads were aligned to the hg38 human reference genome (GRCh38, release v105) using STAR aligner (v.2.7.10a). The resulting BAM files were processed with GATK (v.4.2.4.1) MarkDuplicates, BaseRecalibrator, and ApplyBQSR. RNA base-editing variants were called using GATK HaplotypeCaller. RNA variant loci were compared to those of control samples and filtered based on the following criteria: (1) loci with a read depth of at least 10 were retained; (2) loci with a variant count of at least 2 were retained; (3) loci also present in the control sample were removed; and (4) undeterminable loci due to insufficient sequencing depth in the control sample were excluded. For the replicate-1 experimental sets, untreated replicate-2 was used as the control for filtering, and for the replicate-2 experimental sets, untreated replicate-1 was used as the control. A-to-G edits were counted the number of RNA variant loci with A-to-G edits on the positive strand or T-to-C edits on the negative strand. C-to-T edits were counted the number of RNA variant loci with C-to-T edits on the positive strand or G-to-A edits on the negative strand.

### 7. Cell Lysis for Genomic DNA Analysis

After removing the growth medium, HEK 293T cells were treated with 100 μL of cell lysis buffer (50 mM Tris-HCl; pH 8.0, 1 mM EDTA, 0.005% sodium dodecyl sulfate) supplemented with 5 μL of Proteinase K (Qiagen). The cells were lysed by incubation at 55° C. for 1 h, and then at 95° C. for 10 min. The genomic DNA mixture was subjected to targeted deep sequencing.

### 8. Targeted Deep Sequencing

NGS Libraries for targeted deep sequencing were created using nested PCR. The target area was initially amplified by PCR using PrimeSTAR® GXL polymerase (Takara). Amplicons were amplified again by PCR with TruSeq DNA-RNA CD index-containing primers to label each fragment with adapter and index sequences in order to build NGS libraries. The PCR primers are listed in Table 2 below. The final PCR products were purified with a PCR purification kit (MGmed) and sequenced using a MiniSeq sequencer (Illumina). Base editing frequencies from targeted deep sequencing data were measured with source code (https://github.com/ibs-cge/manud).

TABLE 2

| 1st PCR | Range | 1 st PCR Forward (5′-3′) | 1 st PCR Reverse (5′-3′) |
|---|---|---|---|
| human mtWGS 1 | 2478-10858 | GCAAATCTTACCCCGCCTG | AATTAGGCTGTGGGTGGTTG |
| | (8381 bp) | (SEQ ID NO: 19) | (SEQ ID NO: 20) |
| human mtWGS 2 | 10653-2688 | GCCATACTAGTCTTTGCCGC | GGCAGGTCAATTTCACTGG |
| | (8605 bp) | (SEQ ID NO: 21) | (SEQ ID NO: 22) |
| From 1st PCR | DNA Site | 2nd PCR Forward (5′-3′) | 2nd PCR Reverse (5′-3′) |
| human mtWGS 1 | COX3.1 | TCAATCACCTGAGCTCACCA | ATTAGTTGGCGGATGAAGCA |
| | | (SEQ ID NO: 23) | (SEQ ID NO: 24) |
| human mtWGS 1 | ND1 | AAGGGTGGAGAGGTTAAAGGAG | CCCTGGTCAACCTCAACCTA |
| | | (SEQ ID NO: 25) | (SEQ ID NO: 26) |
| human mtWGS 2 | ND6 | CCACAGCACCAATCCTACCT | TGATTGTTAGCGGTGTGGTC |
| | | SEQ ID NO: 27) | (SEQ ID NO: 28) |

| Nucleus DNA site | 1 st PCR Forward (5′-3′) | 1 st PCR Reverse (5′-3′) |
|---|---|---|
| TYRO3 | CTGTCAACAAAGTGCTGGCC | CTTGACTCCCATGCCTCCTG |
| | (SEQ ID NO: 29) | (SEQ ID NO: 30) |
| Nucleus DNA site | 2nd PCR Forward (5′-3′) | 2nd PCR Reverse (5′-3′) |
| TYRO3 | GTCAACAAAGTGCTGGCC | TTGACTCCCATGCCTCCTG |
| | (SEQ ID NO: 31) | (SEQ ID NO: 32) |

9. RNA Purification from Cultured Mammalian Cells and Targeted RNA Sequencing

RNA was extracted from cultured cells using a NucleoSpin RNA Plus kit (Macherey-Nagel) according to the manufacturer's instructions. RNAs were then reverse transcribed to create cDNAs using a SuperScript IV Reverse Transcriptase Kit (Thermo Fisher), again following the manufacturer's instructions. Regions of interest were then amplified by PCR using the primers shown in Table 3 below. Amplified regions were sequenced using the targeted deep sequencing procedure described above.

TABLE 3

| RNA off-site | 1st PCR Forward (5′-3′) | 1st PCR Reverse (5′-3′) |
|---|---|---|
| OT1- | CCTGACCGTGCTAACTGTGT | TTCATGCTCATTCCCCCCAC |
| chr1: 160214910 | (SEQ ID NO: 33) | (SEQ ID NO: 34) |
| OT2- | GGCGGTACAAGAACAACGAG | GGGGACACACACAGAGAACC |
| chr19: 48630862 | (SEQ ID NO: 35) | (SEQ ID NO: 36) |
| OT3- | AAGTCCCTGGATCATATTCACA | GGATCTTCTGAGCGGGTGT |
| chr14: 102250902 | (SEQ ID NO: 37) | (SEQ ID NO: 38) |
| OT4- | GGTGACAACAGAGCAAAGCA | AGTACATTTCCCCGCTGATAA |
| chr15: 88632045 | (SEQ ID NO: 39) | (SEQ ID NO: 40) |
| OT5- | GGCTTTGACCAGCAACCAAG | TGGACCACACTGACAGGAGA |
| chr17: 59398345 | (SEQ ID NO: 41) | (SEQ ID NO: 42) |
| OT6- | AGCCAAGACCTCACACACAC | AAAACACAGCCCCAGAGAGG |
| chr6: 37175192 | (SEQ ID NO: 43) | (SEQ ID NO: 44) |
| RNA off-site | 2nd PCR Forward (5′-3′) | 2nd PCR Reverse (5′-3′) |
| OT1- | GGAGCTTTTAGGATGTGGGAGT | AGGGAAAGGGAGACTTGGGA |
| chr1: 160214910 | (SEQ ID NO: 45) | (SEQ ID NO: 46) |
| OT2- | CGGCTCAAGGAGAACCAGAT | AGGGAACAGGGCGTAAGTCT |
| chr19: 48630862 | (SEQ ID NO: 47) | (SEQ ID NO: 48) |
| OT3- | CAGGATGTCCTGAAATTAGGG | TGGCGATCTCGTAGAACACA |
| chr14: 102250902 | (SEQ ID NO: 49) | (SEQ ID NO: 50) |
| OT4- | ATCACACTACCCCACCCTCA | ATCTTCCTTCGACCCCCAGA |
| chr15: 88632045 | (SEQ ID NO: 51) | (SEQ ID NO: 52) |
| OT5- | TTTACAGTGGCTTCCGTGGG | TGGACCACACTGACAGGAGA |
| chr17: 59398345 | (SEQ ID NO: 53) | (SEQ ID NO: 54) |
| OT6- | GACGCTTGCTCTGTTTGTGG | AAAACACAGCCCCAGAGAGG |
| chr6: 37175192 | (SEQ ID NO: 55) | (SEQ ID NO: 56) |

### 10. Relative Ratio of DNA On-Target Editing Frequencies to RNA Off-Target Editing Frequencies

To analyze the DNA on-target editing frequency of variants against the RNA off-target editing frequency at six representative sites, the DNA on-target activity and RNA off-target activity of the variants were normalized to the sTALED values. Then, the normalized DNA on-target value is divided by the normalized RNA off-target value, as shown below.

$$Relative ratio ( DNA on - target / RNA off - target ) = \frac{DNA on - target frequency of the variants normalized to wild - type sTALED}{Average of RNA off - target frequency of the variants normalized to wild - type sTALED}$$

In the case of wild-type sTALED, this value is 1 because both the normalized DNA on-target value and RNA off-target value are 1. The higher the relative ratio indicates lower the off-target activity compared to the on-target activity.

### 11. Whole Mitochondrial Genome Sequencing

For whole mitochondrial genome sequencing, three procedures were required: PCR amplification, NGS library creation, and NGS. Initially, cells were treated with 100 μL of cell lysis buffer (50 mM Tris-HCl; pH 8.0, 1 mM EDTA, 0.005% sodium dodecyl sulfate) supplemented with 5 μL of Proteinase K (Qiagen) after removing the growth medium. The cells were lysed by incubation at 55° C. for 1 h, and then at 95° C. for 10 min. Mitochondrial DNA was then amplified by PCR using PrimeSTAR® GXL polymerase (Takara). PCR was performed using two sets of slightly overlapping primers in shown in Table 2 above to reduce primer bias. Each primer pair amplified almost 50% of the mitochondrial DNA. The PCR products were then purified with a PCR purification kit (MGmed). Finally, an Illumina DNA Prep kit with Nextera DNA CD Indexes was used to create an NGS library from the purified PCR products (Illumina). The libraries were then pooled and transferred onto a MiniSeq sequencer (Illumina).

### 12. Analysis of Mitochondrial Genome-Wide Off-Target Editing

To analyze NGS data from whole mitochondrial genome sequencing, a method used to investigate off-target effects in mitochondrial genomes was used. Initially, the Fastq sequences were aligned to the GRCh38 (release v102) reference genome using BWA (v.0.7.17), then created BAM files with SAMtools (v.1.9) by fixing read pairing information and flags. Subsequently, the REDItoolDenovo.py script from REDItools (v.1.2.1) was used to find all thymines and adenines in the mitochondrial genome with conversion rates >0.1%. Positions with conversion rates ≥10% in both treated and untreated samples were identified as SNVs in the cell lines and removed. The construct's on-target sites were excluded. The remaining sites were regarded as off-target sites, and we counted the number of edited A/T nucleotides with an editing frequency >0.1%. The average A/T to G/C editing frequency were calculated for all bases in the mitochondrial genome by averaging the conversion rates at each base location in the off-target sites, as shown below.

$$Average mtDNA - wide A - to - G editing frequency = \frac{Sum of A : T to G : C off - target ( \% )}{All bases in the mitochondrial genome}$$

Mitochondrial genome-wide graphs were constructed by plotting the conversion rates at on-target and off-target sites with an editing frequency ≥1% across the entire mitochondrial genome.

### 13. Cell Viability Assays

Cell viability assays were performed using CellTiter 96® Aqueous One Solution (Promega) at day 2 or day 4 after plasmid transfection. The MTS assay measured the number of viable cells with a colorimetric method. Cells were treated with CellTiter 96® Aqueous One Solution, and the quantification of bio-reduced product was measured by recording the absorbance at 490 nm according to the manufacturer's instructions.

### Example 1: Mitochondrial DNA-Targeted TALEDs Induce Transcriptome-Wide Off-Target Edits

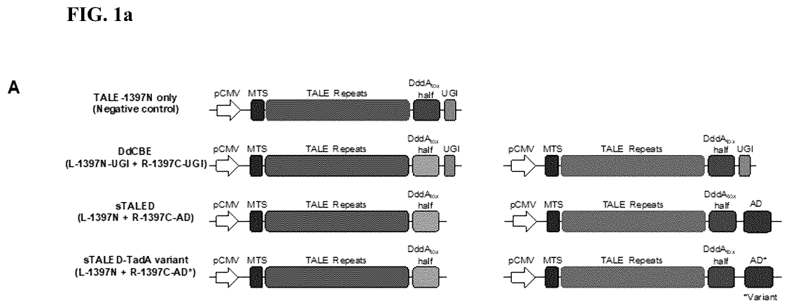

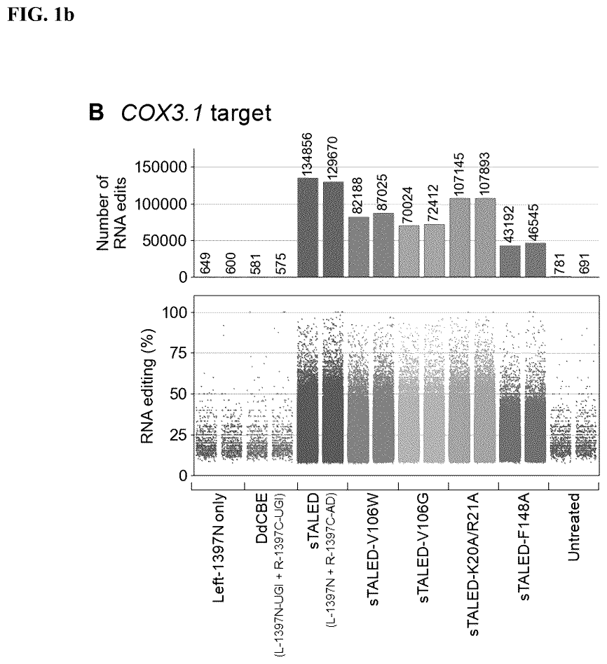

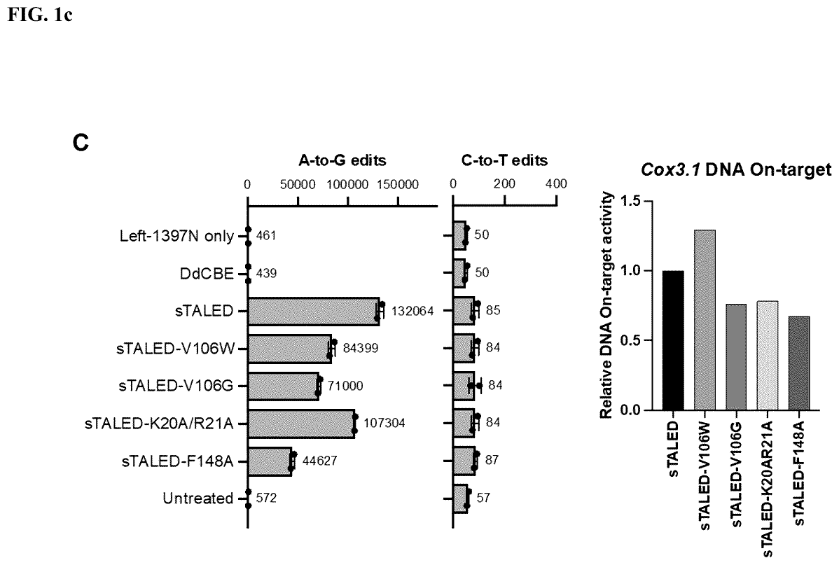

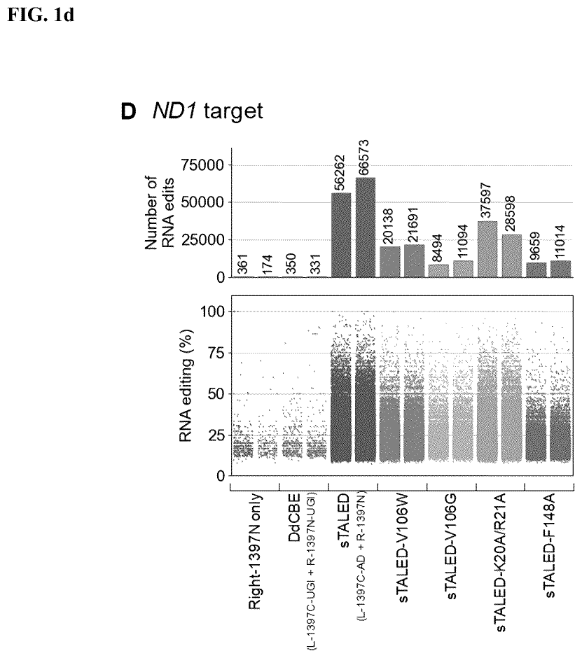

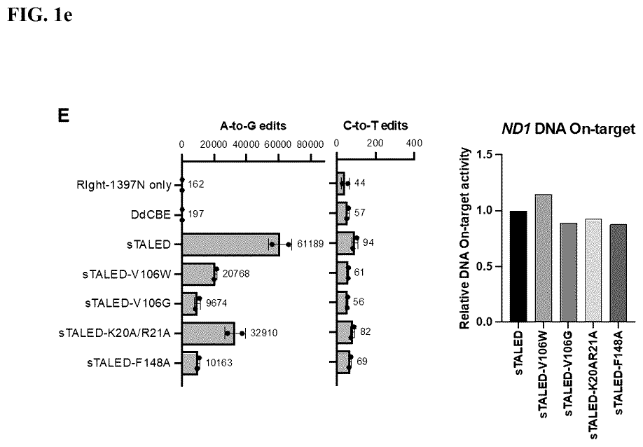

In order to investigate whether the DddA-split TALEDs (sTALEDs) targeted to the COX3.1 or ND1 site could cause unwanted, off-target RNA editing in human embryonic kidney 293T (HEK 293T) cells, transcriptome-wide sequencing of total RNA isolated from cells at day 2 post-transfection was performed with reference to the above Reference Examples. Whole transcriptome sequencing of two independent biological replicates showed that the two sTALEDs, composed of the left- or right-side TALE fused to the N-terminal DddAₜₒₓ half split at G1397 (L-1397N or R-1397N, respectively) and the right- or left-side TALE fused to the C-terminal DddAₜₒₓ half split at G1397 and TadA8e (R-1397C-AD or L-1397C-AD), induced base conversions at >50,000 sites with a frequency of at least 7% (FIG. 1). The vast majority (>99.8%) of these base conversions were A-to-G edits (in cDNA reverse transcribed from RNA) rather than C-to-T edits, indicating that an adenine deaminase rather than a cytosine deaminase was responsible for these single-nucleotide alterations. In contrast, DdCBEs targeted to the same sites or a single sTALED subunit (L-1397N or R-1397N), used as a negative control, did not induce these off-target RNA edits. These results suggest that the other subunit containing TadA8e (L-1397C-AD or R-1397C-AD) was responsible for the observed A-to-G conversions, which resulted from A-to-I alterations in RNA.

To avoid or minimize unwanted, transcriptome-wide off-target A-to-I conversions induced by TALEDs, site-specific mutations were introduced in TadA8e, including V106W, V106G, K20A/R21A (dual mutations), or F148A, which are known to reduce off-target RNA editing when incorporated in CRISPR RNA-guided adenine base editors (ABEs). Transcriptome-wide sequencing showed that sTALED variants incorporating these mutations in TadA8e reduced the number of off-target A-to-G edits significantly but not completely, while retaining DNA on-target editing efficiencies (FIG. 1). Thus, Cox3.1-specific sTALED variants with these site-specific mutations induced RNA off-target edits at sites that numbered from 44,627 (F148A) to 107,304 (K20A/R21A), reducing the number by 18.7% (=(132,064-107,304)/132,064) to 66.2% (=(132,064-44,627)/132,064), compared to the original sTALED (132,064 A-to-G edits) (FIGS. 1(b) and 1(c)). Likewise, ND1-specific sTALED variants reduced the number of RNA off-target edits by at least 46.2% (=(61,189-32,910)/61,189) (K20A/R21A) and up to 84.2% (=(61,189-9,674)/61,189) (V106G) (FIGS. 1(d) and 1(e)). These results show that the same TadA8e variants, used for reducing RNA off-target editing effects of CRISPR RNA-guided ABEs, can also reduce collateral damage caused by sTALEDs. Yet, at least 9,600 and up to 107,000 off-target A-to-G edits persisted even with the best performing TadA8e variants.

### Example 2: Protein Engineering of TALEDs to Avoid RNA Off-Target Edits

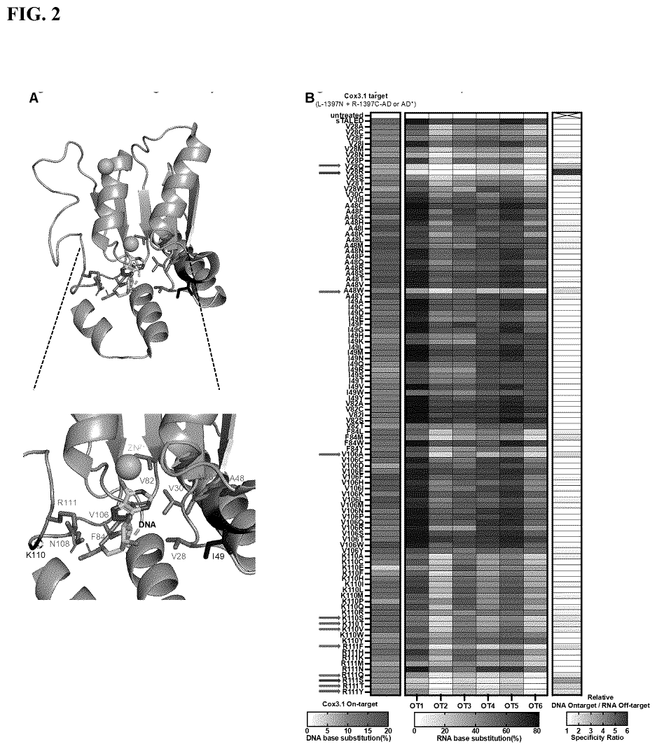

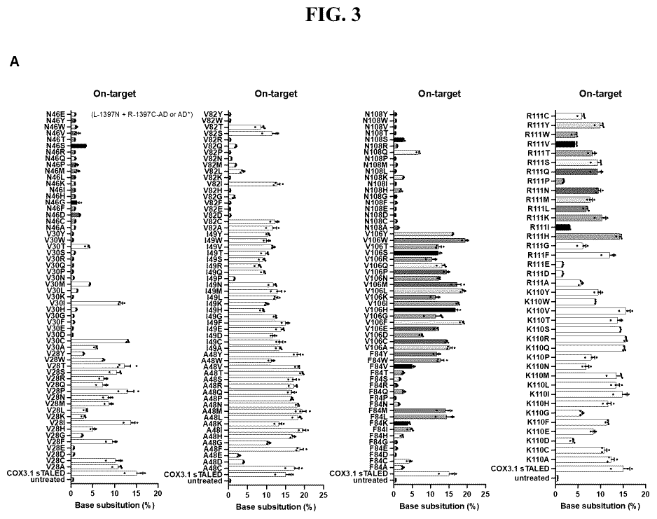

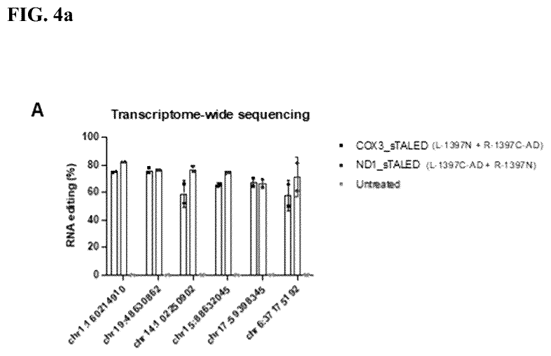

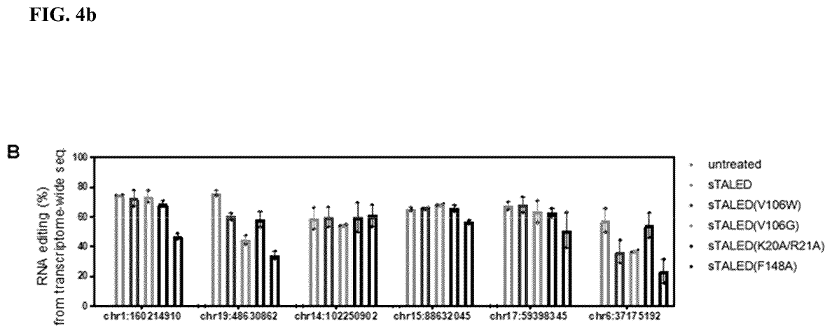

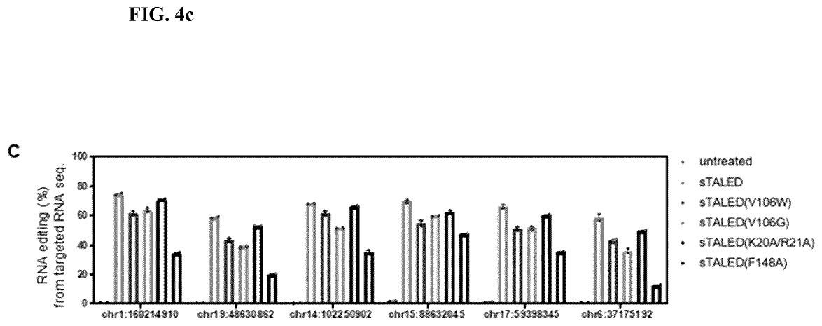

To further minimize transcriptome-wide off-target edits, TALEDs were engineered by mutating amino-acid residues, including V106, at the substrate binding site in TadA8e. To this end, 11 amino-acid residues were selected at the substrate binding site, based on the 3D cryo-electron microscopy structure of ABE8e bound to DNA (FIG. 2(a)), and replaced each of these residues with the other 19 amino-acid residues in the Cox3.1-specific sTALED, making a total of 209 (=11× 19) sTALED variants. As described in Reference Example 3, plasmids encoding each of the resulting sTALED pairs were transfected into HEK 293T cells. Subsequently, on-target editing frequencies at the Cox3.1 site at day 4 post-transfection were measured. Among the 209 STALED variants, a total of 101 sTALED pairs remained highly active in targeted mitochondrial DNA editing with an efficiency of at least 50%, compared to the wild-type sTALED pair (FIG. 3). Then, targeted RNA amplicon sequencing was used to measure off-target editing frequencies of the 101 sTALEDs at six representative sites revealed by the transcriptome-wide sequencing analysis (FIG. 4(a)). These representative sites were mutated with high frequencies that ranged from 60% to 80% by both of the Cox3.1- and ND1-specific TALEDs (FIG. 2(b)). RNA off-target editing frequencies measured by targeted deep sequencing were in good agreement with those estimated by transcriptome-wide sequencing (FIGS. 4(b) and 4(c)). A total of 12 TadA8e variants were chosen, which minimized RNA off-target editing efficiencies at the six representative sites, while retaining mtDNA on-target editing efficiencies (FIG. 2(b)).

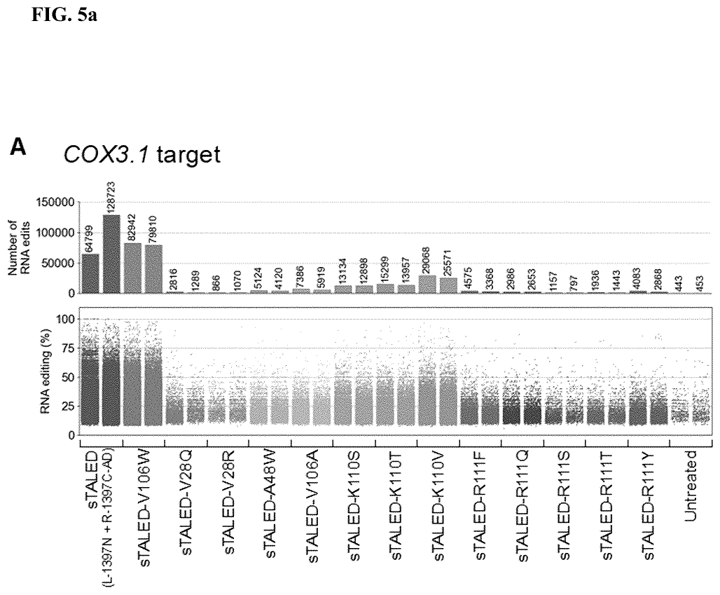

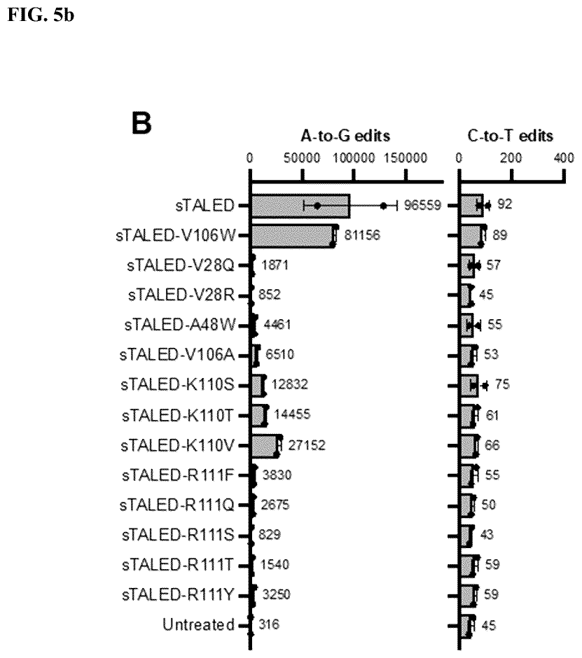

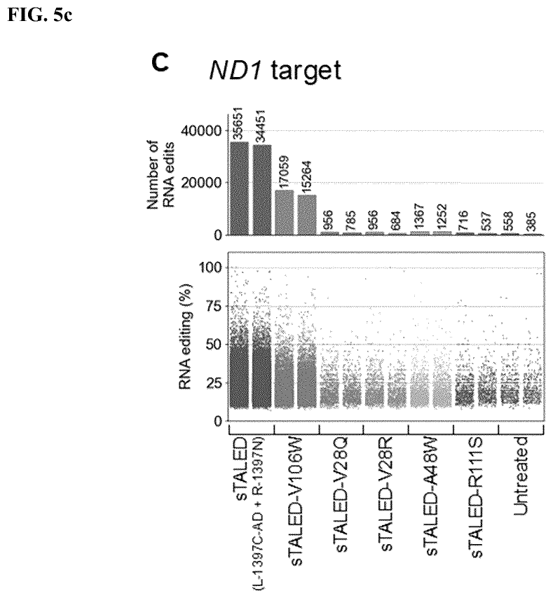

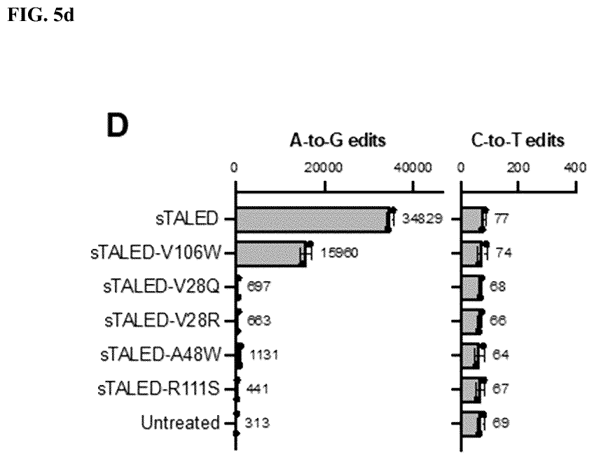

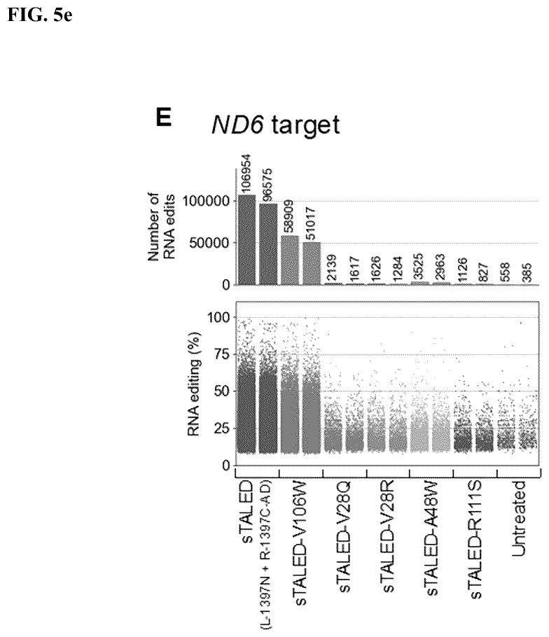

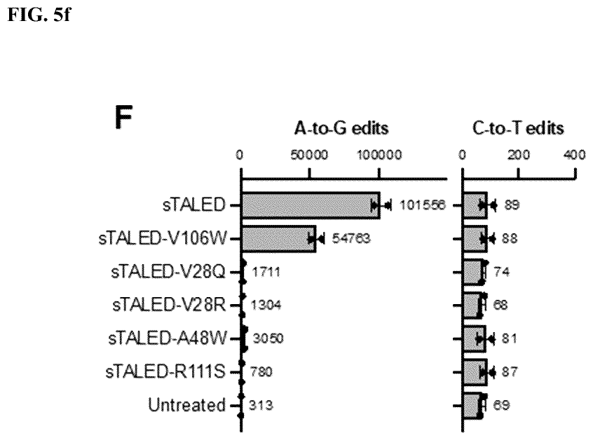

Transcriptome-wide sequencing was performed to investigate whether the 12 sTALED variants could avoid off-target editing at sites other than the six representative sites (FIG. 5). All of these variants reduced the number of off-target edits dramatically. For example, sTALED variants with V28R and R111S induced off-target edits at merely 852 and 829 sites, respectively, whereas the original sTALED and sTALED-V106W (STALED with the V106W mutation in TadA8e) induced off-target edits at 96,559 and 81,156 sites, respectively (FIGS. 5(a) and 5(b)). Thus, sTALED-V28R and -R111S avoided >99% of RNA off-target edits. Considering that 316 A-to-G edits were found in the untreated DNA sample used as a negative control, which were likely caused by high-throughput sequencing errors, these results showed that these sTALED variants almost completely avoided RNA off-target editing.

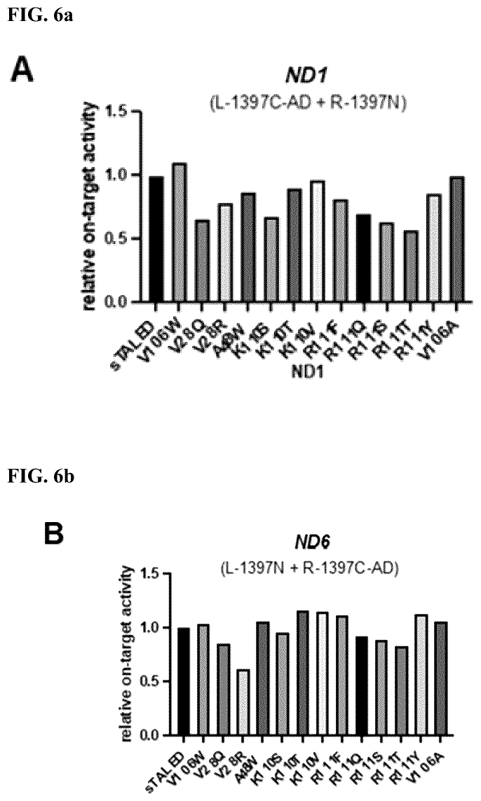

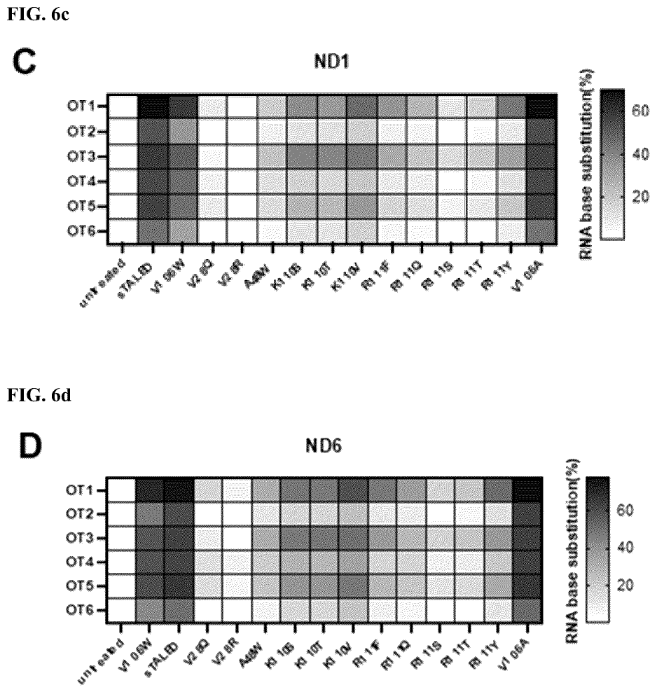

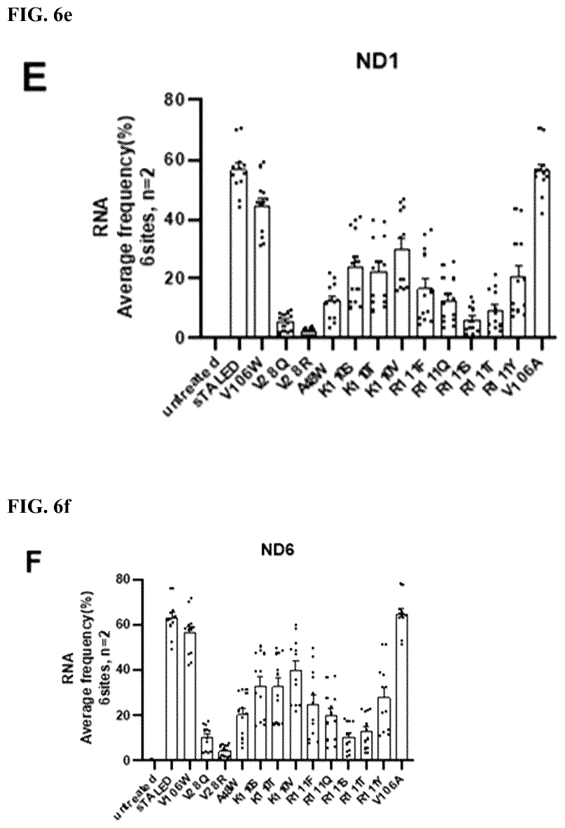

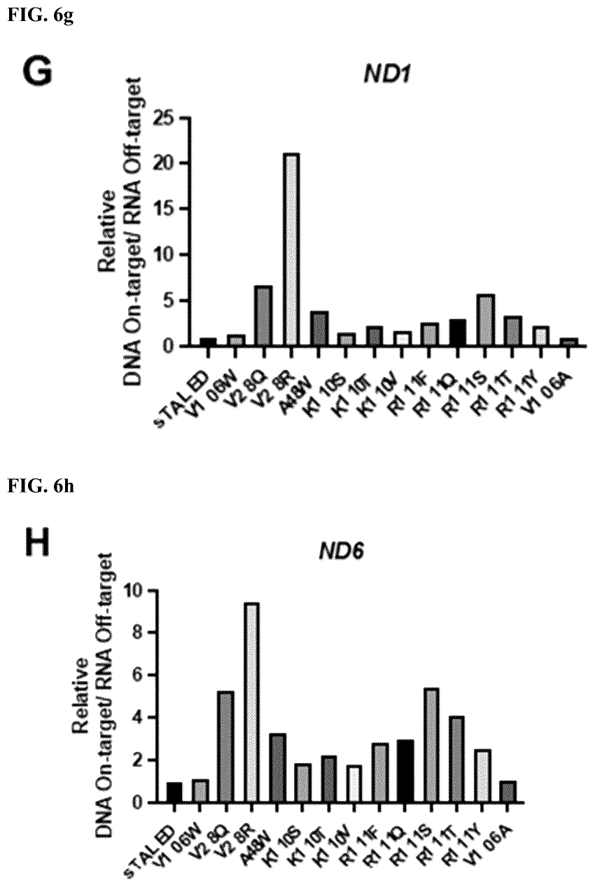

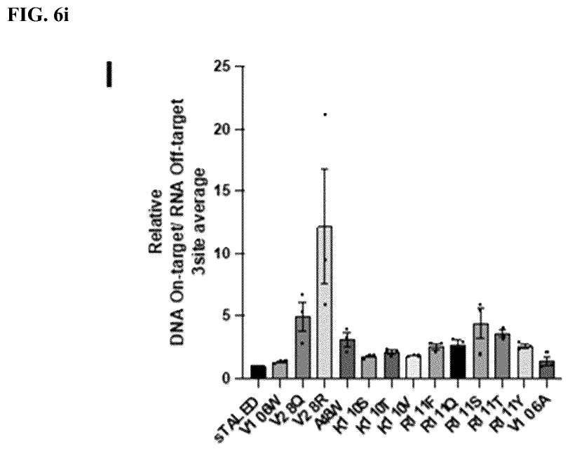

In addition, it was further investigated whether these TadA8e mutations could reduce RNA off-target editing when incorporated in sTALEDs targeted to other mitochondrial DNA target sites (FIGS. 5C-F and FIG. 6). Targeted RNA amplicon sequencing showed that most of the ND1- and ND6-specific sTALED variants reduced RNA off-target editing frequencies at the six representative sites significantly (FIGS. 6(c)-(f)). Mitochondrial DNA on-target editing frequencies were also measured via targeted deep sequencing (FIGS. 6(a) and 6(b)) and then the ratio of the DNA on-target editing frequency to the RNA off-target editing frequency was obtained (FIGS. 6(g) and 6(h)). Based on these results, the following four TadA8e variants, V28Q, V28R, A48W, and R111S, were selected which exhibited high mitochondrial DNA on-target activity and low RNA off-target activity, compared to the original sTALED and sTALED-V106W targeted to the Cox3.1, ND1, and ND6 sites (FIG. 6(i)). Whole transcriptome sequencing showed that ND1- and ND6-specific sTALED variants with each of these four variations reduced the number of RNA off-target edits drastically (FIG. 5), in line with the results shown above with the Cox3.1-specific STALED variants. Again, both the ND1- and ND6-specific sTALED-V28R or-R111S variants were the most discriminatory, inducing the fewest RNA off-target edits (FIGS. 5(c)-(f)).

### Example 3: Engineered TALEDs Reduce Bystander and Off-Target Editing

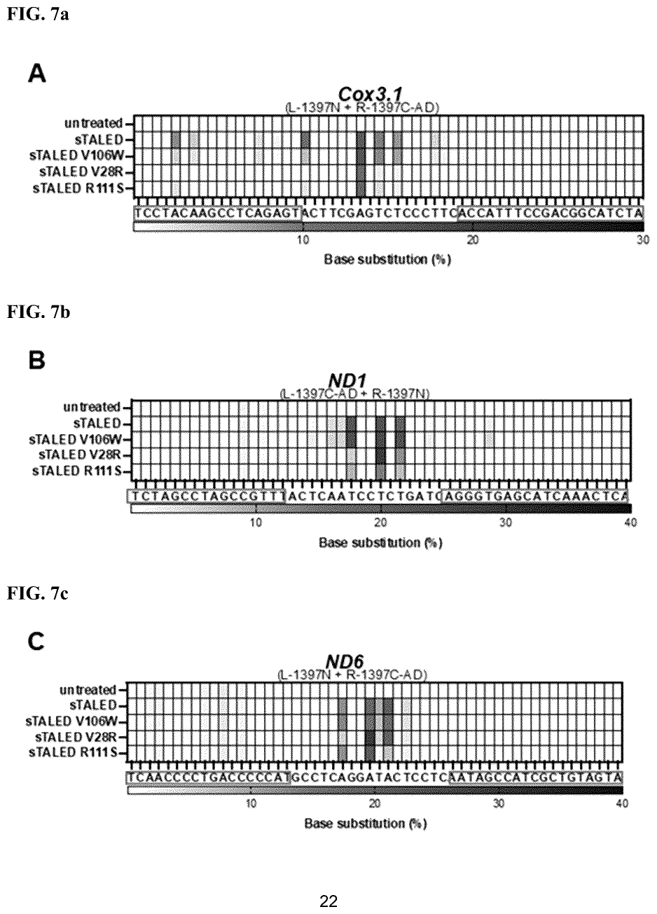

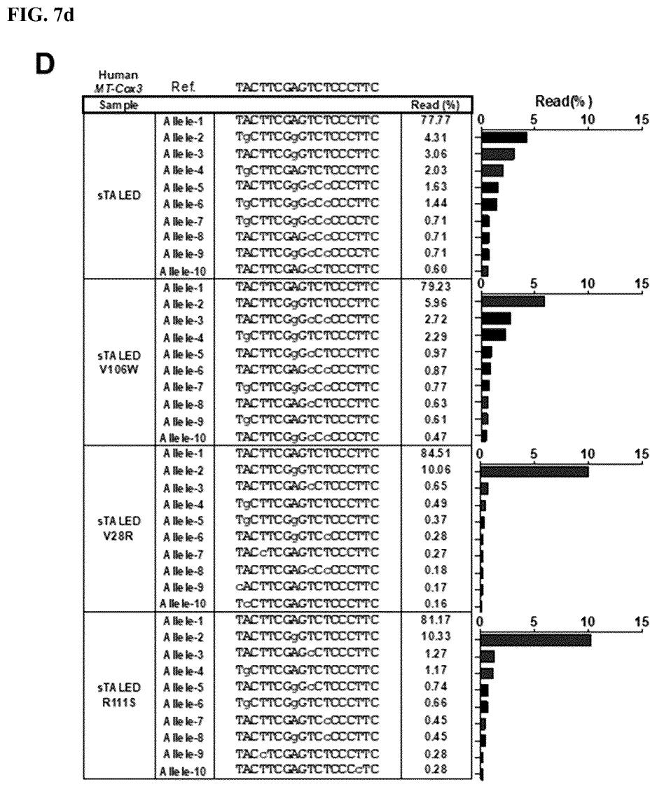

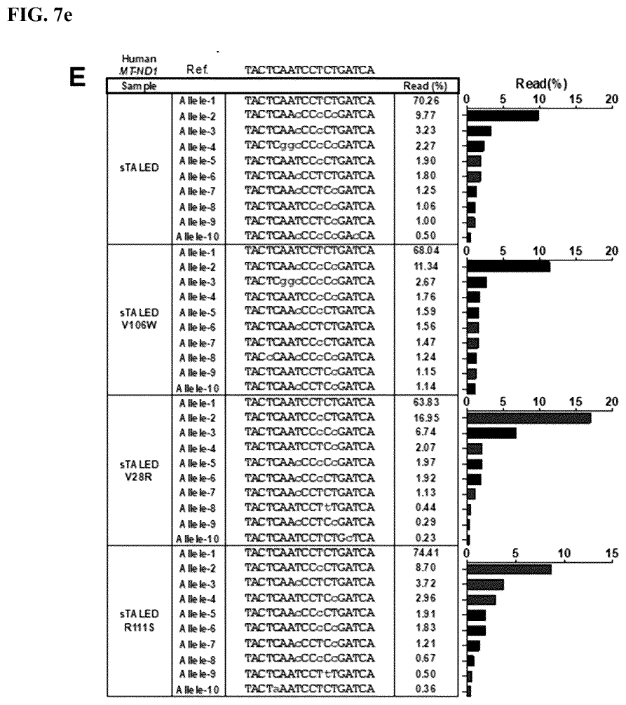

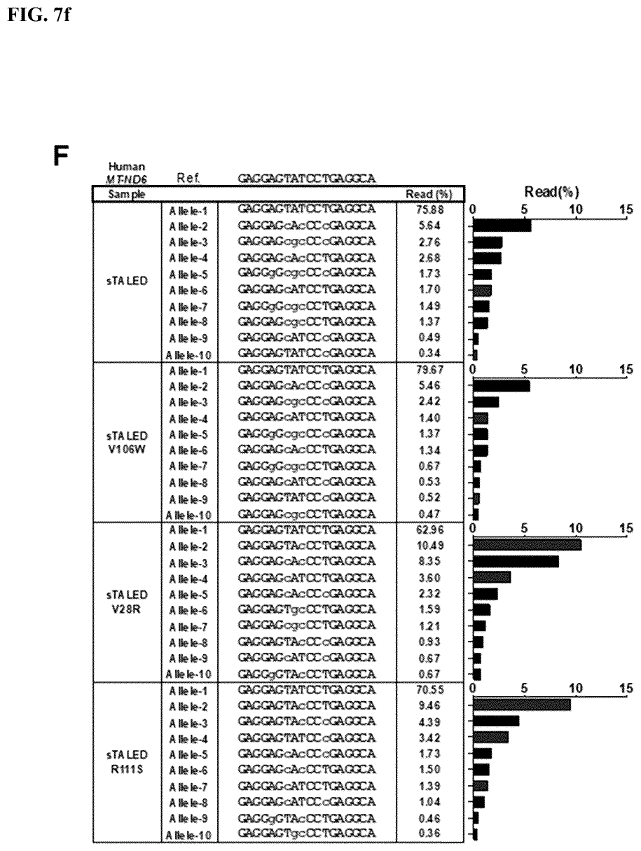

STALED variants with site-specific mutations at the TadA8e substrate-binding site could also reduce bystander editing at the target site and off-target editing in the mitochondrial genome, because these mutations could potentially fine-tune adenine deaminase activity for DNA substrates in addition to reducing activity for RNA substrates. That said, base editing frequencies at each nucleotide position were examined. Both sTALED-V28R and -R111S variants specific to the Cox3.1, ND1, and ND6 sites induced A-to-I edits in a narrower window than did the wild-type STALEDs and sTALED-V106W variants (FIG. 7). For example, the wild-type sTALED and STALED-v106W targeted to the Cox3.1 site induced A-to-I edits at multiple positions, with frequencies of >1.1%, not only in the spacer region between the two TALE-binding sites but also in the TALE-binding sites. In contrast, the Cox3.1-specific sTALED-V28R induced an A-to-I edit at a single position in the spacer region (FIG. 7a).

Next, the frequencies of edited alleles induced by these sTALEDs targeted to the three mitochondrial DNA sites were compared. Most of the mutant alleles induced by the original STALEDs or sTALED-V106W variants contained multiple-base edits rather than single-base edits. In sharp contrast, sTALED-V28R and -R111S induced mutant alleles with single-base substitutions much more frequently than multiple-base substitutions. Thus, the most abundant alleles with a single A-to-I edit in the middle of the spacer region were induced with low frequencies of 3.06% (Cox3.1), 1.90% (ND1), and 1.70% (ND6) by the original sTALEDs, whereas the same alleles were induced with high frequencies of 10.1% (Cox3.1), 17.0% (ND1), and 10.5% (ND6) by the sTALED-V28R variants (FIGS. 7(d)-(f)). This result has an important implication for the use of TALEDs in disease modeling as well as therapeutic applications. TALEDs inducing single-base substitutions with no or few bystander edits are desired, because the vast majority of pathogenic mitochondrial DNA mutations, responsible for mitochondrial genetic disorders, are single-nucleotide variations rather than multiple-nucleotide variations.

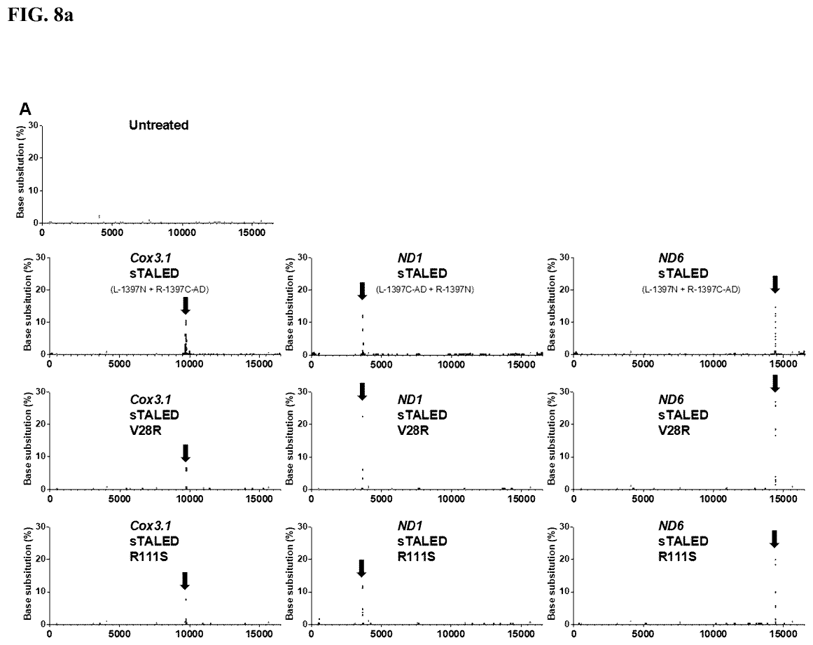

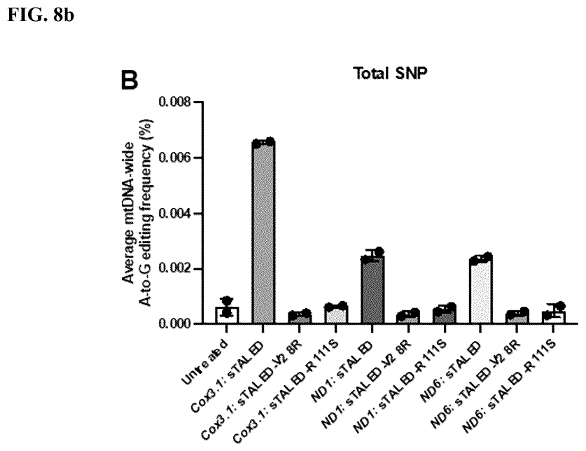

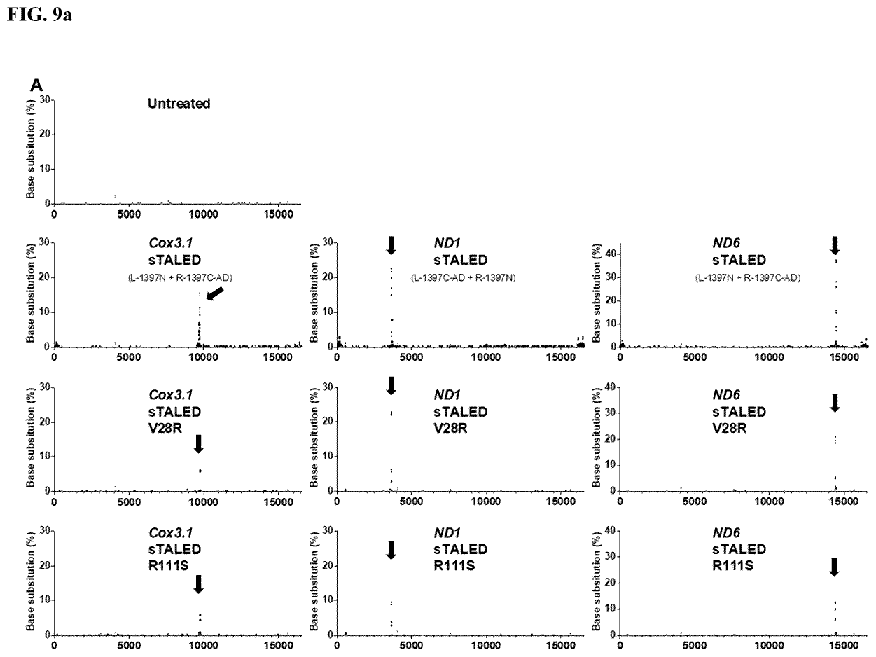

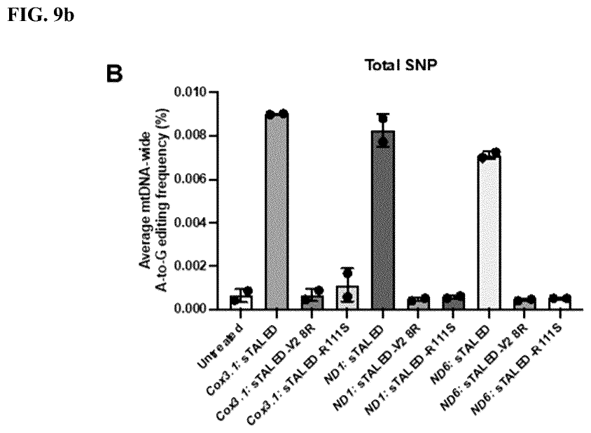

In addition, whole mitochondrial genome sequencing was performed to assess and compare off-target effects of the wild-type sTALEDs and sTALED variants at day 4 post-transfection (FIG. 8). The wild-type sTALEDs targeted to the three mitochodrial DNA sites induced off-target mutations with average frequencies of mitochondrial genome-wide off-target editing that ranged from 0.0024% to 0.0066%, 4~10-fold higher than that observed in the untreated control (0.0006%). In contrast, sTALED-V28R and R111S induced off-target edits with average frequencies of <0.0006%, similar to the baseline frequency observed in the negative control (FIG. 8B). Furthermore, the number of off-target edits induced in the mitochondrial genome was also reduced by the sTALED variants. Thus, the ND1-specific, wild-type sTALED caused A-to-I off-target edits at 108 sites in human mitochodrial DNA with frequencies of >0.1%, whereas sTALED-V28R and -R111S induced off-target edits at 14 and 17 sites, respectively, similar to the baseline number (that is, 24) seen in the untreated sample (FIG. 8(a)). Whole mitochondrial genome sequencing at day 2 post-transfection was also performed (FIG. 9), when RNA off-target edits were induced at the highest level. The original sTALEDs targeted to the three sites induced off-target edits with average frequencies that ranged from 0.0071% to 0.0090%, 11~14-fold higher than that observed in the untreated control (0.0006%). However, the V28R and R111S variants did not induce off-target edits, compared to the negative control (FIG. 9(b)).

### Example 4: Time Course Measurements of DNA On-Target and RNA Off-Target Editing Frequencies

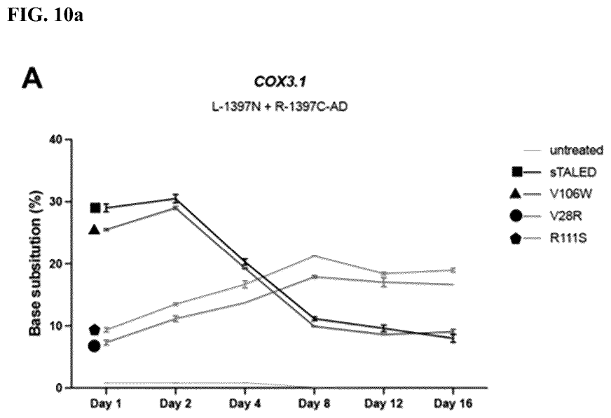

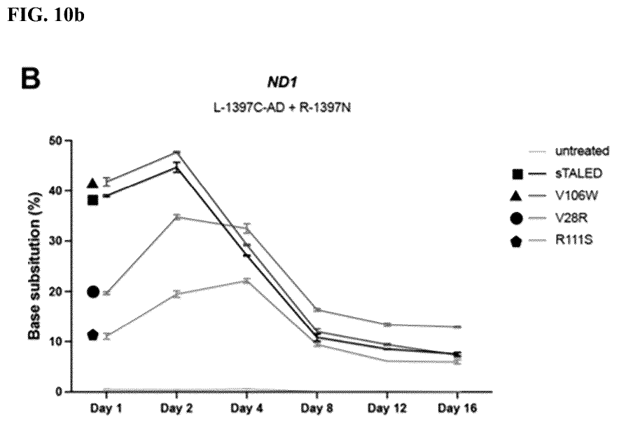

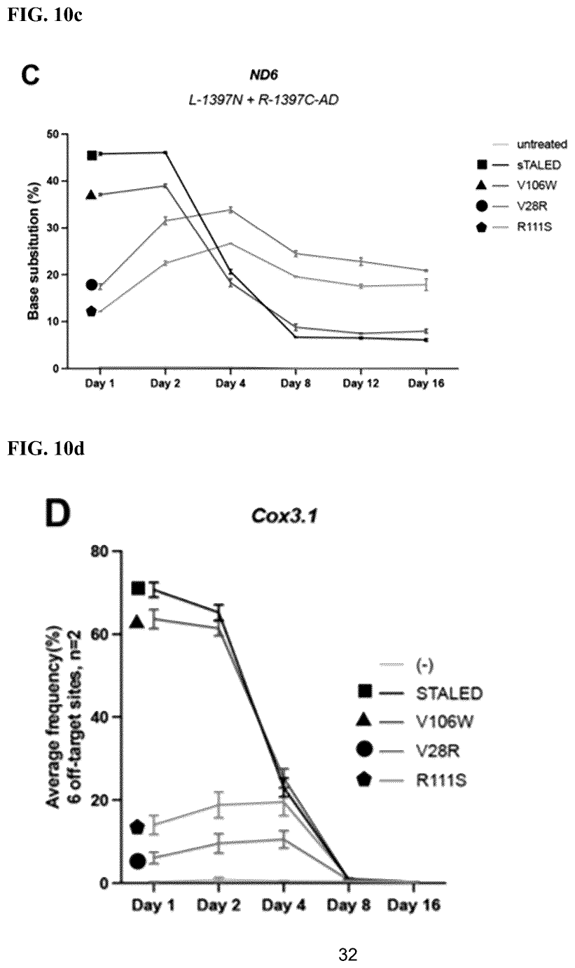

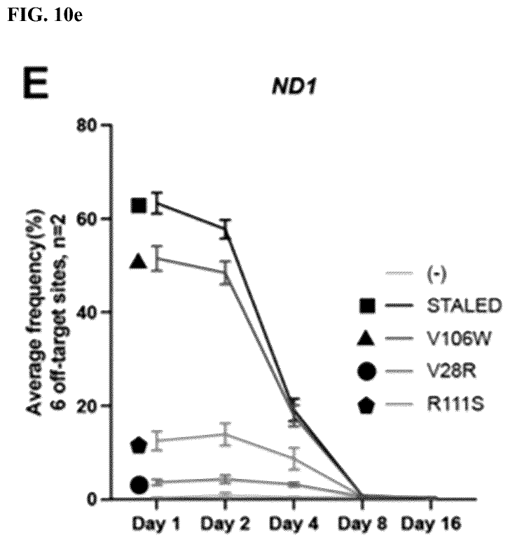

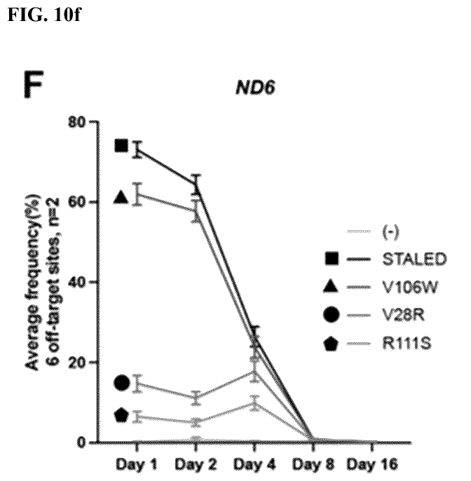

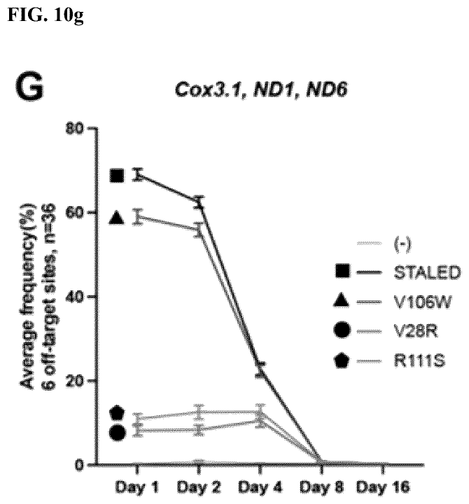

In FIG. 10, it was investigated whether on-target mutations induced by various forms of STALEDs were stably maintained and how long RNA off-target variations persisted over time. Using targeted deep sequencing, DNA on-target editing frequencies at the three mitochondria DNA sites (FIG. 10(a)-(c)), and RNA off-target editing frequencies at the six representative sites (FIG. 10(d)-(g)), co-edited by sTALEDs targeted to the three sites, were measured at various time points. RNA edits were heavily induced by sTALEDs and sTALED-V106W variants at day 1 and 2 post-transfection but almost completely disappeared by day 8 post-transfection. The new STALED variants reduced RNA off-target editing frequencies by several fold, compared to sTALEDs or the sTALED-V106W variants, even at day 1 and 2 post-transfection (FIG. 10(g)). Mitochondria DNA on-target edits induced by the sTALEDs and sTALED-V106W variants were not stably maintained. Thus, the frequencies of mitochondria DNA on-target edits induced by the STALEDs and sTALED-V106W variants dropped significantly over time. For example, mitochondria DNA on-target edits were induced by the ND6-specific sTALED and sTALED-V106W with high frequencies of up to 47% and 40%, respectively, at day 1 and 2 post-transfection but with low frequencies of <10% at day 8 or later (FIG. 10(c)). In contrast, the frequencies of on-target edits induced by sTALED-V28R and -R111S increased or were more stably maintained over time. As a result, the editing frequencies observed for our new sTALED variants were lower at day 1 and 2 post-transfection but were higher at day 8 or later than those induced by previous versions of sTALEDs (FIG. 10(a)-(c)).

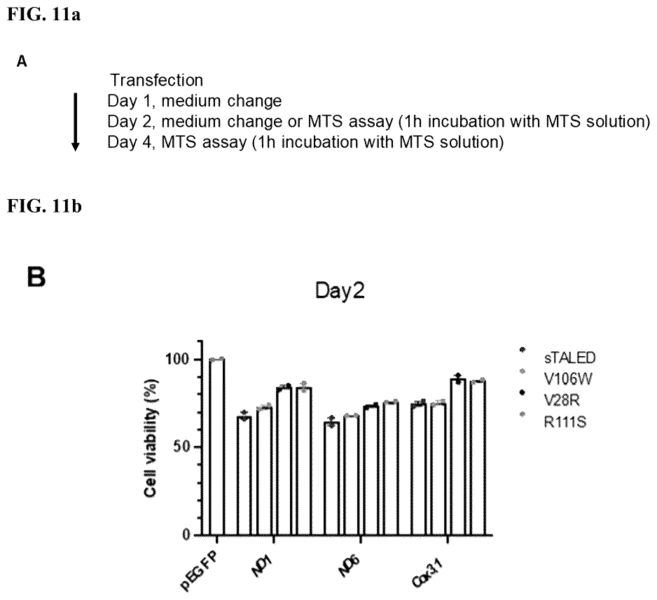

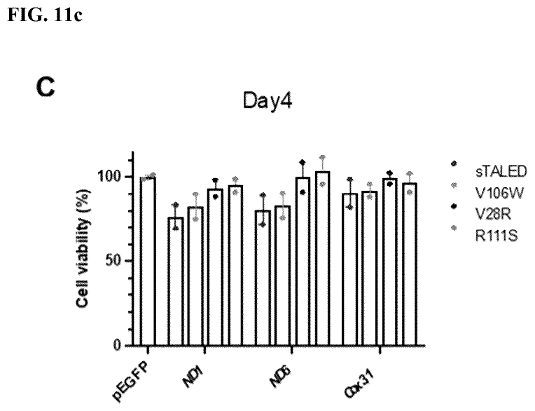

Based on these results, it could be concluded that the sTALEDs and sTALED-V106W variants were cytotoxic, because they induced too many RNA off-target edits with high frequencies, and that mtDNA-edited cells could not divide or died out over time. However, STALED variants of the present invention could be tolerated, possibly because they avoided RNA off-target editing or did not induce too many bystander edits at the target site and off-target mutations in the mitochondrial genome. Further, MTS cell proliferation assays (FIG. 11(a) were performed to confirm that the sTALEDs and sTALED-V106W variants were indeed cytotoxic, reducing cell viability significantly, compared to the negative control (pEGFP transfection) (FIG. 11(b)). The sTALED variants were tolerated much better, such that cell viability was not reduced at day 4 post-transfection (FIG. 11(c)).

### Example 5: TadA8e-V28R and -R111S Incorporated in CRISPR RNA-Guided ABEs

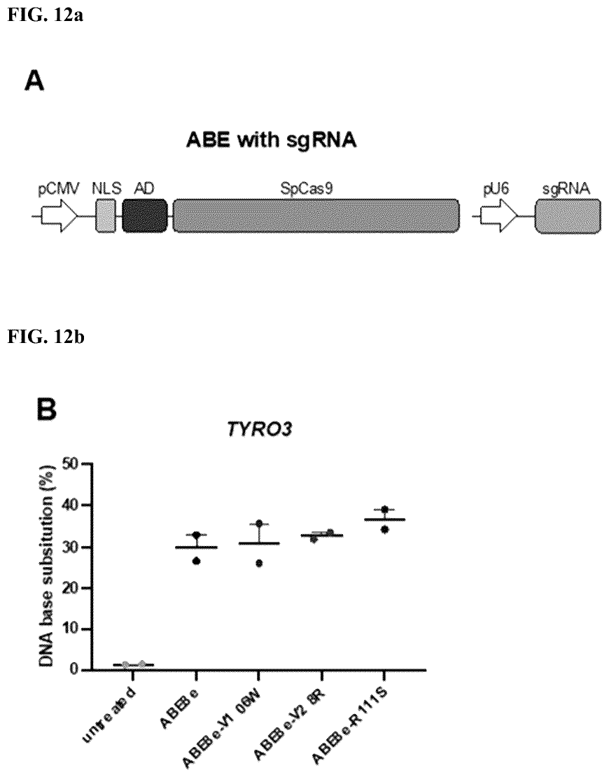

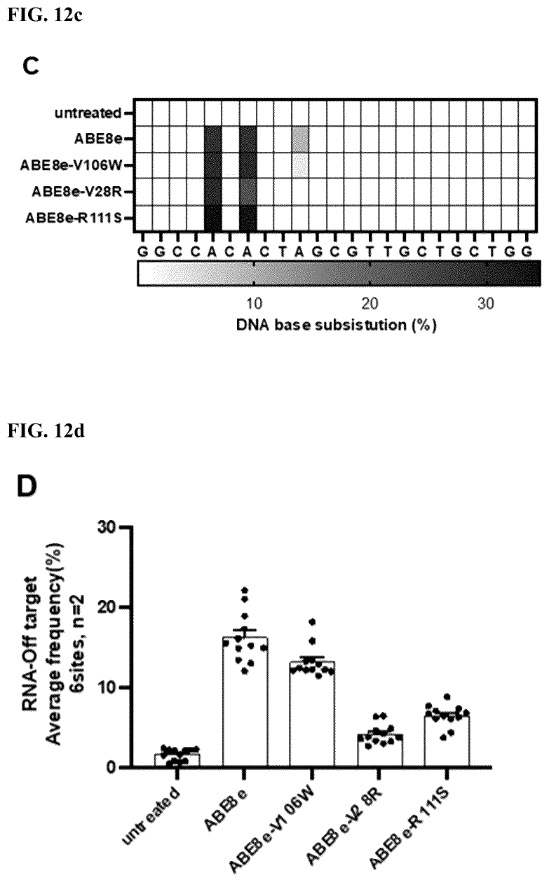

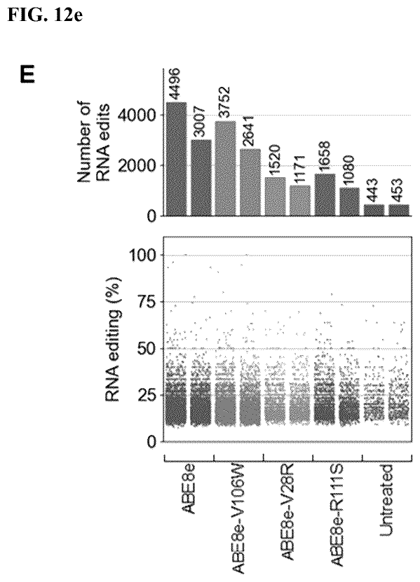

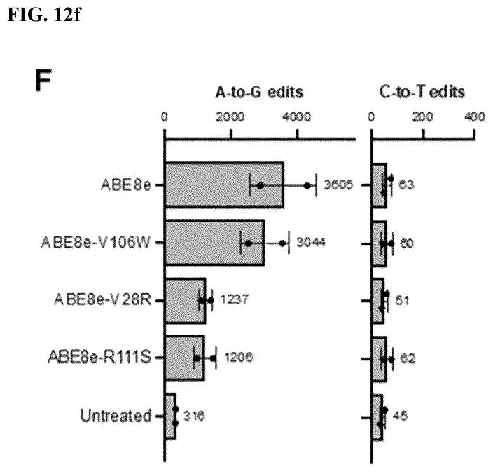

In FIG. 12, it was investigated whether the V28R and R111S mutations in TadA8e could also reduce bystander editing and RNA off-target editing induced by CRISPR RNA-guided ABEs, widely used for nuclear DNA editing. First, on-target and bystander editing frequencies at the TYRO3 site were measured. Both ABE8e-V28R and -R111S were as efficient as ABE8e and ABE8e-V106W (ABE8eW) at the target site with editing frequencies of >30% (FIGS. 12(b) and 12(c)). The V28R and R111S variants exhibited a narrowed editing window with efficient editing (up to 37%) at positions 5 and 7 (A5 and A7 in FIG. 12(c)) in the proto-spacer region but with almost no bystander editing (0.8% and 0.5%, respectively) at position 10 (A10). In contrast, ABE8e and ABE8eW showed a broader editing window with maximum editing at A5 (29% and 30%, respectively) and A7 (29% and 30%, respectively) and substantial bystander editing at A10 (9.3% and 3.1%, respectively).

Next, targeted RNA amplicon sequencing was used to assess RNA off-target editing activities. ABE8e-V28R and -R111S reduced average RNA off-target editing frequencies measured at a total of 6 sites by 3.8 fold and 2.5 fold, respectively, compared to ABE8e, and 3.1 fold and 2.0 fold, compared to ABE8eW (FIG. 12(d)). Furthermore, whole transcriptome sequencing showed that ABE8e-V28R and -R111S reduced the number of RNA off-target edits substantially, compared to ABESe and ABE8eW (FIGS. 12(e) and 12(f)). These results show that the two new TadA8e variations, V28R and R111S, can also improve ABE8e by reducing RNA off-target edits and bystander edits.

### Example 6: DNA On-Target and RNA Off-Target Editing by mTALEDs and sTALEDs

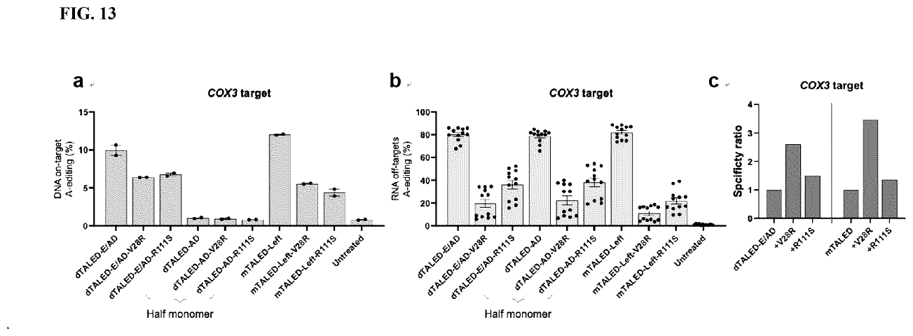

In FIG. 13, it was investigated whether dimeric TALEDs (dTALED) and monomeric TALEDs (mTALED) variants with V28R and R111S in addition to sTALED could reduce RNA off-target editing. In the dTALED, each TALE unit contains a cytosine deaminase (e.g. DddAtex variant (E1347A)) on one side and an adenine deaminase (e.g. TadA8e) on the other side. The mTALED, on the other band, is characterized by both cytosine deaminase and adenine deaminase being present in a single TALE unit.

In FIG. 13(a), it was confirmed that both dTALEDs and mTALEDs targeted to Cox3 site exhibited on-target activity. Furthermore, it was observed that gene editing did not occur when only the adenine deaminase was present in the TALE unit. FIG. 13(b) relates to the results of editing efficiency at six representative sites. Similar to the sTALEDs, a significant reduction in RNA off-target efficiencies for both dTALEDs and mTALEDs variants with V28R and R11IS was observed compared to the TadA8e.

In FIG. 13(c), the specificity ratios of RNA off-target editing relative to on-target editing induced by Cox3-specific TALEDs were analyzed. The results demonstrate that the TadA variants with V28R and R11IS, not only reduced the RNA off-target efficiency in sTALED and other systems like ABE but also in dTALEDs and mTALEDs as well. These results indicate that RNA off-target effects were predominantly induced by the TadA adenine deaminase rather than by a DNA binding proteins or cytosine deaminase. Moreover, the TadA variants can remarkably reduce such undesired RNA off-target effects.

From the above description, it will be understood by those skilled in the art that the present invention may be embodied in other specific forms without departing from the spirit or essential characteristics thereof. In this regard, it should be understood that the above-described embodiments are to be considered in all respects as illustrative and not restrictive. The scope of the present invention should be construed as being included in the scope of the present invention without departing from the scope of the present invention as defined by the appended claims.

What is claimed is:

1. An adenine deaminase comprising the amino acid sequence of SEQ ID NO:1 or an amino acid sequence having at least 80% sequence homology to the amino acid sequence of SEQ ID NO: 1, wherein at least one amino acid residue selected from residues 28, 30, 46, 48, 49, 82, 84, 106, 108, 110, and 111 of SEQ ID NO:1 or the corresponding amino acid residue of the amino acid sequence having at least 80% sequence identity to the amino acid sequence of SEQ ID NO:1 is substituted with another amino acid.

2. The adenine deaminase according to claim 1, wherein the substitution is at least one selected from the group consisting of:
V28Q, or V28R,
A48W,
F84M,
V106A,
K110S, K110T, or K110V, and
R111F, R111Q, R111S, R111T, or R111Y,
of the amino acid sequence of SEQ ID NO:1.

3. The adenine deaminase according to claim 2, wherein the substitution is at least one selected from the group consisting of:
V28Q, or V28R,
A48W, and
R111S,
of the amino acid sequence of SEQ ID NO:1.

4. A fusion protein comprising:
a DNA-binding protein; and
the modified adenine deaminase according to claim 1.

5. The fusion protein according to claim 4, wherein the substitution in the modified adenine deaminase is at least one selected from the group consisting of:
V28Q, or V28R,
A48W,
F84M,
V106A,
K110S, K110T, or K110V, and
R111F, R111Q, R111S, R111T, or R111Y,
of the amino acid sequence of SEQ ID NO:1.

6. The fusion protein according to claim 5, wherein the substitution in the modified adenine deaminase is at least one selected from the group consisting of:
V28Q, or V28R,
A48W, and
R111S,
of the amino acid sequence of SEQ ID NO:1.

7. The fusion protein according to claim 4, wherein the DNA-binding protein is zinc finger protein, TALE (transcription activator-like effector), or CRISPR-associated nuclease.

8. The fusion protein according to claim 7, wherein the DNA-binding protein is TALE.

9. The fusion protein according to claim 8, wherein the TALE is a dual TALE module consisting of a first TALE module and a second TALE module.

10. The fusion protein according to claim 4, wherein the DNA-binding protein is a Cas nickase or a Cas protein lacking endonuclease activity.

11. The fusion protein according to claim 4, wherein the fusion protein further comprises a cytosine deaminase.

12. The fusion protein according to claim 11, wherein the cytosine deaminase is in a full length form or in a two-split form.

13. The fusion protein according to claim 11 or 12, wherein the cytosine deaminase is APOBEC (apolipoprotein B editing complex), AID (activation-induced deaminase), DddAₜₒₓ (cytosine deaminase specific to double-stranded DNA), or a variant thereof.

14. The fusion protein according to any one of claims 4-12, wherein the fusion protein further comprises UGI (uracil glycosylase inhibitor).

15. The fusion protein according to any one of claims 4-12, wherein the DNA is nuclear DNA or organellar DNA.

16. The fusion protein according to claim 15, wherein the fusion protein further comprises NLS (nuclear localization signal).

17. The fusion protein according to claim 15, wherein the fusion protein further comprises MTS (mitochondrial targeting sequence) or CTP (chloroplast transit peptide).

18. The fusion protein according to claim 15, wherein the fusion protein further comprises NES (nuclear export signal).

19. The fusion protein according to claim 4, wherein the fusion protein further comprises a nickase.

20. The fusion protein accordingly to claim 19, wherein the fusion protein comprises a first fusion protein comprising the DNA-binding protein and the modified adenine deaminase, and a second fusion protein comprising the DNA-binding protein and the nickase.

21. The fusion protein according to claim 4, wherein the nickase is MutH, a MutH variant, or Nt.BspD6I(C).

22. A polynucleotide encoding the modified adenine deaminase according to any one of claims 1 to 3, or the fusion protein according to any one of claims 4-12 and 19-21.

23. An expression vector comprising the polynucleotide according to claim 22.

24. A base editing composition for A-to-G base editing in DNA comprising the fusion protein according to any one of claims 4-10 and 19-21, a polynucleotide encoding the fusion protein or an expression vector comprising the polynucleotide.

25. The base editing composition according to claim 24, wherein the fusion protein further comprises a cytosine deaminase.

26. The base editing composition according to claim 25, wherein the cytosine deaminase is present in two split form, and the fusion protein comprises a first fusion protein comprising a first split of the cytosine deaminase and a second fusion protein comprising a second split of the cytosine deaminase.

27. The base editing composition according to claim 26, wherein the first split comprises the amino acid sequence of SEQ ID NO: 9 or 11, and the second split comprises the amino acid sequence of SEQ ID NO: 10 or 12.

28. The base editing composition according to claim 24, wherein the composition exhibits a reduced off-target effect involving an unintended base alteration in DNA and/or RNA.

29. A method for A-to-G base editing in DNA comprising delivering the base editing composition according to claim 24 to a cell containing a target DNA.

30. The method according to claim 29, wherein the delivery is carried out ex vivo.

31. The method according to claim 29, wherein the delivery is carried out in vivo.

32. The method according to claim 29, wherein the target DNA is nuclear DNA, organellar DNA, or mitochondrial DNA of a human subject with a hereditary disease.

33. The method according to claim 29, wherein the method induces base editing at a single nucleotide residue in the target DNA with a frequency of at least 10%.

34. The method according to claim 29, wherein the method exhibits reduced off-target editing effects compared to when using a base editor comprising an adenine deaminase comprising the amino acid sequence of SEQ ID NO: 1, wherein the off-target editing effects are characterized by unintended base alterations in DNA or RNA.

35. The method according to claim 29, wherein the method exhibits reduced off-target editing effects compared to when using a base editor comprising an adenine deaminase comprising the amino acid sequence of SEQ ID NO: 1 with V106W mutation, wherein the off-target editing effects are characterized by unintended base alterations in DNA or RNA.

36. The method according to claim 29, wherein the method induces base editing at a single nucleotide residue in the target gene with a frequency of at least 10%.

37. A method for reducing off-target effect, the method comprising delivering the base editing composition according to claim 24 to a cell containing a target DNA, wherein the method exhibits reduced off-target editing effects compared to when using a base editor comprising an adenine deaminase comprising the amino acid sequence of SEQ ID NO: 1, wherein the off-target editing effects are characterized by unintended base alterations in DNA or RNA.
